(19) United States
(12) Patent Application Publication
(10) Pub. No.: US 2021/0214303 A1
(43) Pub. Date: Jul. 15, 2021

(54) SCORPION VENOM BENZOQUINONE DERIVATIVES AND USES THEREOF

(71) Applicants: The Board of Trustees of the Leland Stanford Junior University, Stanford, CA (US); UNIVERSIDAD NACIONAL AUTONOMA DE MEXICO, Cludad de Mexico, Coyacan (MX); INSTITUTO NACIONAL DE CIENCIAS MEDICAS Y NUTRICION SALVADOR ZUBIRAN, Cludad de Mexico, Alcaldia Tlalpan (MX)

(72) Inventors: Richard Neil ZARE, Stanford, CA (US); Jose Ignacio VEYTIA-BUCHELI, San Luis Potosi (MX); Edson Norberto Carcamo NORIEGA, Cuernavaca (MX); Gnanamani ELUMALAI, Stanford, CA (US); Shyam SATHYAMOORTHI, Stanford, CA (US); Lourival Domingos POSSANI POSTAY, Cuernavaca (MX); Shibdas BANERJEE, Howrah (IN); Rogelio Enrique HERNANDEZ PANDO, Ciudad de Mexico (MX)

(21) Appl. No.: 17/058,962

(22) PCT Filed: May 20, 2019
(86) PCT No.: PCT/US2019/033055

§ 371 Date: Nov. 25, 2020

Related U.S. Application Data
(60) Provisional application No. 62/678,156, filed on May 30, 2018.

(51) Int. Cl. C07C 323/22; C07C 319/20; A61P 35/02; A61P 31/04

(52) U.S. Cl. CPC C07C 323/22; A61P 31/04; A61P 35/02; C07C 319/20

(57) ABSTRACT

Provided are colored 1,4-benzoquinone compounds obtained by oxidation of precursor molecules from the venom of the scorpion Diplocentrus melici (Diplocentridae family). Schemes for the chemical synthesis of these compounds using reagents commercially available are also provided. Biological assays show that the red compound (3,5-dimethoxy-2-(methylthio)cyclohexa-2,5-diene-1,4-dione) is very effective at killing Staphylococcus aureus and that the blue compound (5-methoxy-2,3-bis(methylthio)cyclohexa-2,5-diene-1,4-dione) has remarkable activity against Mycobacterium tuberculosis. The blue compound is effective against multi-drug-resistant tuberculosis (MDR-TB) and is not detrimental to lung epithelium. Both compounds were found to be cytotoxic to human neoplastic cell lines and to mononuclear cells (PBMCs).

### CROSS REFERENCE TO RELATED APPLICATIONS

This application claims the benefit of U.S. Provisional Application No. 62/678,156 titled “SCORPION VENOM BENZOQUINONE DERIVATIVES AND USES THEREOF” filed May 30, 2018 which is incorporated herein by reference in its entirety.

### STATEMENT REGARDING FEDERALLY SPONSORED RESEARCH

This invention was made with government support under grant number AFOSR FA9550-16-1-0113 awarded by the United States Air Force. The government has certain rights in the invention.

### TECHNICAL FIELD

The present disclosure is generally related to 1,4 benzoquinones synthesized from scorpion venom precursors. The present disclosure further generally relates to the use of methods of synthesizing the 1,4 benzoquinones. The present disclosure further relates to the use of the 1,4 benzoquinones as antimicrobial and anticancer agents.

### BACKGROUND

Worldwide, scorpion stings are a significant source of morbidity and mortality, annually disabling approximately 1.5 million humans (Chippaux J. P. (2012) Drug design, Develop. Therapy 6: 165-173). Initial investigations into scorpion venom were focused on isolating and structurally characterizing poisonous compounds. Such studies inspired the development of effective antivenom therapies, mainly antibodies generated in hyper-immunized horses (Espino-Solis et al., (2009) J. Proteomics 72: 183-199). The majority of these toxin compounds are peptides that interfere with Na⁺, K⁺, Ca²⁺ and Cl⁻ ion-channels in target tissues (Cahalan M. D. (1975) J. Physiol. 244: 511-534; Catterall W. A. (1975) Proc. Nat. Acad. Sci. U.S.A. 72:1782-1786; Rochat et al., (1979) Adv. Cytopharmacol. 3: 325-334; Miranda et al., (1970) Euro. J. Biochem 16(3):514-523; Possani et al., (2000) Biochimie 82: 861-868).

Not all compounds in scorpion venom are injurious to human health. Indeed, in recent years, compounds with a variety of beneficial properties, including antibacterial (Torres-Larios et al., (2000) Euro. J. Biochem. 267: 5023-5031), antimalarial (Conde et al., (2000) FEBS Letts 471: 165-168), and anti-inflammatory activity (Gurrola et al. (2012) Biochem. 51: 4049-4061) have been isolated (Ortiz et al., (2015) Toxicon 93: 125-135). Most venom components described thus far are small peptides and large proteins. Isolation of non-proteinic components is an area of emerging research (Banerjee et al., (2018). J. Natural Prods. 81: 1899-1904).

Globally, there are over 2300 different scorpion species; although the venom of only about 1% has been characterized (Santibanez-Lopez et al., (2015) Toxins 8(1)). Within just Mexico, there are at least 281 different species of scorpions. Scorpions of the Centruroides genus, Buthidae family, are dangerous to humans and have been well-studied. Among the 20 different known families of scorpions, there are some, including the Diplocentridae family, for which there is no analysis of the venom.

### SUMMARY

The present disclosure encompasses non-naturally occurring 1,4-benzoquinones obtained by oxidation of precursor molecules found in the venom of the scorpion Diplocentrus melici. Initially a viscous colorless liquid, the extracted venom colors within minutes under ambient conditions. From this colored mixture, two compounds, one red, the other blue, were isolated. The red compound A is 3,5-dimethoxy-2-(methylthio)cyclohexa-2,5-diene-1,4-dione and the blue compound B is 5-methoxy-2,3-bis(methylthio)cyclohexa-2,5-diene-1,4-dione. Synthesis schemes for compounds A and B are also provided by the disclosure. In vitro, the red and blue 1,4 benzoquinones are effective anti-proliferative agents against Staphylococcus aureus, whereas the blue 1,4 benzoquinone is more active against Mycobacterium tuberculosis, including against a multi-drug resistant (MDR) strain. The bactericidal activity against these pathogens of both 1,4 benzoquinones is comparable to that of available antibiotics. The blue 1,4 benzoquinone was also effective in vivo with mouse models of MDR tuberculosis infection. Both of the 1,4-benzoquinones of the disclosure are cytotoxic to a variety of mammalian neoplastic cell lines.

One aspect of the disclosure, therefore, encompasses embodiments of a 1,4-benzoquinone having the structure:

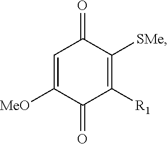

wherein R₁ can be a methylthio group or an alkoxy group.

In some embodiments of this aspect of the disclosure, the 1,4-benzoquinone can have a structure according to Formula A:

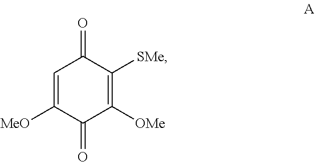 [SMILES: COC1=CC(=O)C(SC)=C(OC)C1=O]

or a derivative thereof.

In some embodiments of this aspect of the disclosure, the 1,4-benzoquinone can have a structure according to Formula B:

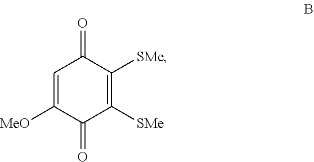 [SMILES: COC1=CC(=O)C(SC)=C(SC)C1=O]

or a derivative thereof.

Another aspect of the disclosure encompasses embodiments of a pharmaceutical formulation comprising: a 1,4-benzoquinone having the structure:

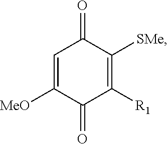

wherein R₁ is a methylthio group or an alkoxy group; and

a pharmaceutically acceptable carrier.

Still another aspect of the disclosure encompasses embodiments of a method of synthesizing a 1,4-benzoquinone, wherein the 1,4-benzoquinone can have a structure according to Formula A:

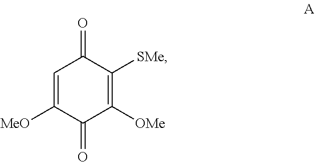 [SMILES: COC1=CC(=O)C(SC)=C(OC)C1=O]

and wherein the 1,4-benzoquinone can be synthesized according to Scheme A:

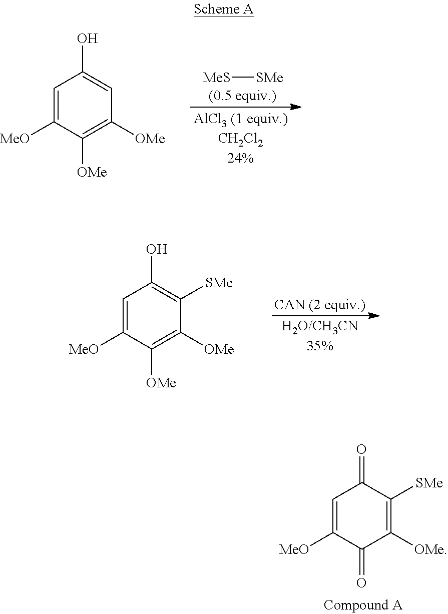

Still another aspect of the disclosure encompasses embodiments of a method of synthesizing a 1,4-benzoquinone, wherein the 1,4-benzoquinone can have a structure according to Formula B:

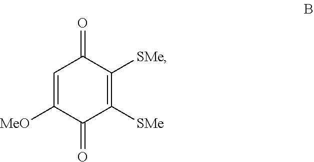 [SMILES: COC1=CC(=O)C(SC)=C(SC)C1=O]

and wherein the 1,4-benzoquinone is synthesized according to Scheme B:

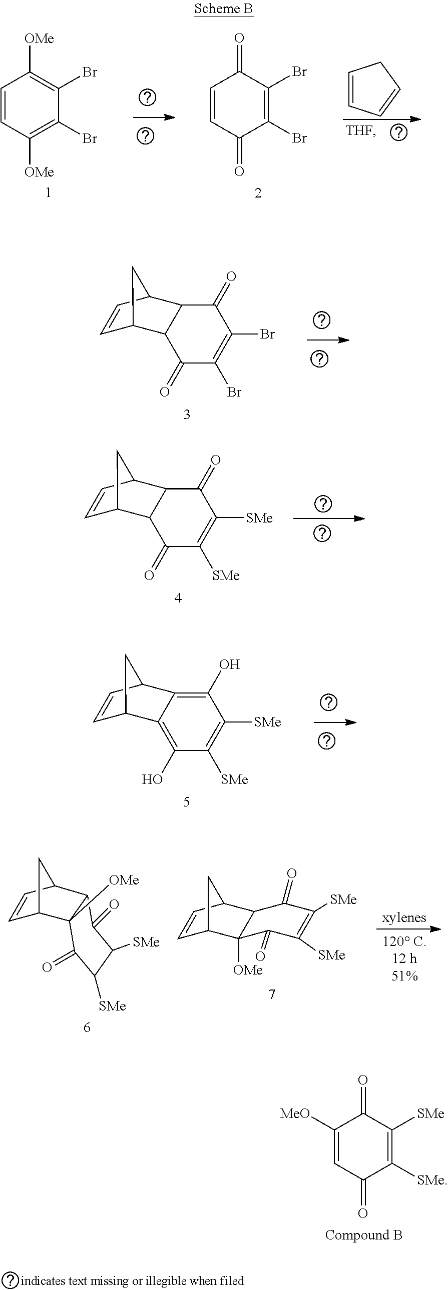

Yet another aspect of the disclosure encompasses embodiments of a method of reducing the proliferation of a bacterial species, the method comprising the step of contacting a population of a bacterial species with an amount of a 1,4-benzoquinone having a structure:

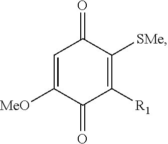

wherein R₁ can be a methylthio group or an alkoxy group and for a period sufficient to reduce the proliferation of the bacterial species.

In some embodiments of this aspect of the disclosure, the bacterial species can be a Staphylococcus or a Mycobacterium.

In some embodiments of this aspect of the disclosure, the bacterial species can be a Staphylococcus aureus or a Mycobacterium tuberculosis.

In some embodiments of this aspect of the disclosure, the 1,4-benzoquinone can be administered to an animal or human subject having a bacterial infection.

In some embodiments of this aspect of the disclosure, the 1,4-benzoquinone can be administered to the animal or human subject in a pharmaceutically acceptable formulation comprising the 1,4-benzoquinone and a pharmaceutically acceptable carrier.

Still another aspect of the disclosure encompasses embodiments of a method of treating a bacterial infection in an animal or human subject, the method comprising: administering to the animal or human subject a pharmaceutically acceptable formulation comprising a 1,4-benzoquinone having the structure:

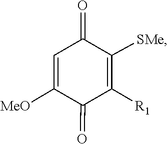

wherein R₁ is a methylthio group or an alkoxy group; and a pharmaceutically acceptable carrier.

In some embodiments of this aspect of the disclosure, the 1,4-benzoquinone has a structure according to Formula A:

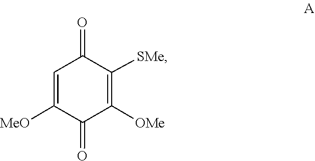 [SMILES: COC1=CC(=O)C(SC)=C(OC)C1=O]

or a derivative thereof.

In some embodiments of this aspect of the disclosure, the bacterial infection can be a Staphylococcal infection.

In some embodiments of this aspect of the disclosure, the bacterial infection can be a Staphylococcus aureus infection.

In some embodiments of this aspect of the disclosure, the 1,4-benzoquinone can have a structure according to Formula B:

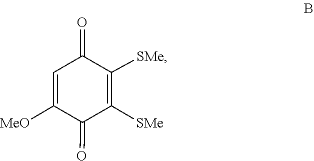 [SMILES: COC1=CC(=O)C(SC)=C(SC)C1=O]

or a derivative thereof.

In some embodiments of this aspect of the disclosure, the bacterial infection can be a Mycobacterial infection.

In some embodiments of this aspect of the disclosure, the bacterial infection can be a Mycobacterium tuberculosis infection.

Another aspect of the disclosure encompasses embodiments of a method of reducing the proliferation of a population of cancer cells, the method comprising the step of contacting a population of cancer cells with an amount of a 1,4-benzoquinone having the structure:

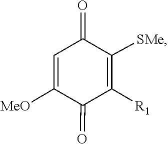

wherein R₁ is a methylthio group or an alkoxy group and for a period sufficient to reduce the proliferation of the cancer cells.

In some embodiments of this aspect of the disclosure, the population of cancer cells is a tumor or a non-tumor cancer.

In some embodiments of this aspect of the disclosure, the population of cancer cells can be a non-tumor cancer, wherein the non-tumor cancer is a leukemia.

In some embodiments of this aspect of the disclosure, the 1,4-benzoquinone can be administered to the animal or human subject in a pharmaceutically acceptable formulation comprising the 1,4-benzoquinone and a pharmaceutically acceptable carrier.

In some embodiments of this aspect of the disclosure, the 1,4-benzoquinone can have a structure according to Formula A or Formula B:

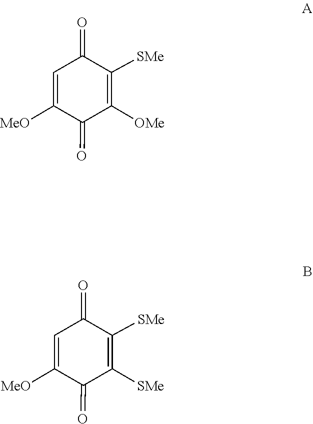 [SMILES: COC1=CC(=O)C(SC)=C(OC)C1=O ; COC1=CC(=O)C(SC)=C(SC)C1=O]

or a derivative thereof.

### BRIEF DESCRIPTION OF THE DRAWINGS

Further aspects of the present disclosure will be readily appreciated upon review of the detailed description of its various embodiments, described below, when taken in conjunction with the accompanying drawings.

FIG. 1 illustrates the derivatization of the venom of Diplocentrus melici. Freshly extracted venom (Left) was exposed to air for about 10 minutes, during which time the color became dark red (Right).

FIGS. 2A and 2B illustrate the chromatographic purification of the red and blue colored compounds from the oxidized venom of Diplocentrus melici. First, red-colored soluble venom was separated into 3 fractions by gel filtration in Sephadex G50 (FIG. 2A). Fraction FI contained most of the proteinic material of the venom. Red-colored fractions FII and FIII were separated by RP-HPLC (FIG. 2B). Further purification of fractions FII and FIII yielded pure samples of the red and blue compounds. Peaks corresponding to the compounds of interest are indicated with a star.

FIG. 3A illustrates the purification of the scorpion-produced precursor to the colored compounds. Fresh venom from Diplocentrus melici was resuspended in acetone and the resulting extract was separated by RP-HPLC. From this, six main peaks were identified. Peaks 4 and 6 corresponded to the red and blue compounds, respectively. All other peaks were collected and bubbled with air for 2 hours. At the end of this time, the compounds in peaks 2 and 5 had completely transformed into the red and blue compounds, respectively.

FIG. 3B illustrates that the precursor compounds did not inhibit the growth of Staphylococcus aureus in a disk-diffusion assay. There was no appearance of color on the disks or in the agar during the incubation time.

FIG. 3C illustrates a disk-diffusion assay of red and blue 1,4 benzoquinones showing inhibitory activity against Staphylococcus aureus. Ampicillin (5 μg) was used as a positive control.

FIG. 3D illustrates the determination of the minimal inhibitory concentrations (MICs) of the red and blue 1,4 benzoquinones against Staphylococcus aureus. The MICs determined by the broth microdilution assay are 4 μg/mL for the red 1,4 benzoquinone and 6 μg/mL for the blue 1,4 benzoquinone. Ampicillin was used as a positive control. Each result is reported as the mean±SD.

FIGS. 4A and 4B illustrate that after fractions corresponding to peaks 2 and 5 were bubbled with air, their contents were converted into compounds with retention times and absorbance spectra (insets) identical to the red and blue 1,4 benzoquinones.

FIG. 5 illustrates high-resolution positive-ion mode ESI mass spectrum of the red compound showing: (Panel A) protonated, sodiated, and potassiated ion signals; (Panel B) isotopic distribution of the protonated ion signals. Inset of (B) suggests the empirical formula, C₉H₁₀O₄S, which corresponds well with the theoretical m/z and isotopic distribution of the protonated species (Panel C). The lower panel table suggests very high mass accuracy (0.28 ppm) of the proposed formula.

FIG. 6 illustrates high-resolution positive-ion mode ESI mass spectrum of the blue compound showing: (Panel A) protonated, sodiated, and potassiated ion signals; (Panel B) isotopic distribution of the protonated ion signals. Inset of (B) suggests the empirical formula, C₉H₁₀O₃S₂, which corresponds well with the theoretical m/z and isotopic distribution of the protonated species (Panel C).

FIG. 7 illustrates the collision induced dissociation tandem mass spectrometry with (CID-MS/MS) data of the red compound protonated species (m/z 215.0368) showing the neutral loss of CO, CH₃OH and CH₃SH, indicating the presence of carbonyl, methoxy, and methylthio functional groups in the analyte molecule.

FIG. 8 illustrates the CID-MS/MS data of the blue compound protonated species (m/z 231.0142) show the neutral loss of CO, CH₃OH and CH₃SH, indicating the presence of carbonyl, methoxy, and methylthio functional groups in the analyte molecule.

FIG. 9 illustrates the Heteronuclear Multiple Bond Correlation (HMBC) spectrum between carbons and protons (upper panel) proposes the structure of the red compound as shown in the inset. The key HMBC correlations are shown by red arrows in the structure. The lower panel tabulates the chemical shifts of protons and carbons obtained from ¹H NMR and HMBC experiments. The δ value of the C6 carbon was obtained from HSQC experiment (spectrum not shown).

FIG. 10 illustrates the HMBC spectrum between carbons and protons (upper panel) proposes the structure of the blue compound as shown in the inset. The key HMBC correlations are shown by red arrows in the structure. The lower panel tabulates the chemical shifts of protons and carbons obtained from ¹H NMR and HMBC experiments. The δ value of the C⁶ carbon was obtained from HSQC experiment.

FIG. 11 illustrates the precursor compounds found in peaks 2 and 5 are likely hydroquinones.

FIG. 12 illustrates a comparison of the retention time in RP-HPLC using an analytic C18 column between native (N) and synthetic (S) blue (A) and red (B) 1,4 benzoquinones. Samples of synthetic and native benzoquinones showed the same chromatographic behavior with retention times around 32.6 and 25.2 min for the blue and the red benzoquinones, respectively. A mixture of synthetic and native benzoquinones showed only one main peak.

FIG. 13A illustrates the cytotoxic effect of the red compound on neoplastic cell lines. Cells were treated with the 1,4 benzoquinone for 12 hours and cell death was evaluated by the release of the stable cytosolic enzyme lactate dehydrogenase. Each result is reported as the mean±SD.

FIG. 13B illustrates the cytotoxic effect of the blue compound on neoplastic cell lines. Cells were treated with the 1,4 benzoquinone for 12 hours and cell death was evaluated by the release of the stable cytosolic enzyme lactate dehydrogenase. Each result is reported as the mean±SD.

FIG. 14A illustrates a cytotoxic activity assay of the red and blue 1,4 benzoquinones. Hemolysis was evaluated using fresh human erythrocytes. Lysis was evaluated by the absorbance of the supernatant at 415 nm after 2 hours of incubation with different concentrations of the 1,4 benzoquinones. Triton X-100 was used as positive control. Neither 1,4 benzoquinone showed hemolytic activity at all concentrations tested.

FIG. 14B illustrates a cytotoxic effect of the 1,4 benzoquinones of the disclosure on peripheral blood mononuclear cells (PBMCs). PBMCs were incubated with the red and blue 1,4 benzoquinones at a concentration of 25 μM for 12 hours, and cell death was evaluated by the release of the stable cytosolic enzyme lactate dehydrogenase. Both 1,4 benzoquinones were found to be cytotoxic against PBMCs. Each result is reported as the mean±SD.

FIG. 15 illustrates an assay of glutathione oxidation in the presence of the blue and red 1,4 benzoquinones. Different concentrations of 1,4 benzoquinones (in phosphate buffer) were reacted with a solution of 120 μM GSH. After one hour of reaction, 200 μM of Ellman's reagent (5,5′dithiobis-(2-nitrobenzoic acid, DTNB) was added and the resulting glutathione-DTNB conjugate was visualized at 412 nm.

FIG. 16A illustrates the detection of intracellular reactive oxygen species (ROS) using the oxidant-sensing probe dichloro-dihydro-fluorescein diacetate (DCFH-DA). TE671 muscle cells were used. Cells were cultured according to the ATCC guidelines. Before treatment, cells were incubated for one hour with a 10 μM solution of DCFH-DA. After the cells were washed 3×PBS, either the red or blue 1,4 benzoquinone was administered at a concentration of 25 μM. Cells were then incubated at 37° C. in 5% CO₂ for 6 hours. Following the incubation period, cells were harvested and evaluated with a Zeiss Axioskop fluorescence microscope using a 485-nm excitation and 530-nm emission filter combination. Intracellular oxidation of the DCFH-DA in cells treated with the 1,4 benzoquinones was observed in dark-ground. Treatment with hydrogen peroxide (50 μM) was used as positive control.

FIG. 16B illustrates that the incubation with either the red or blue 1,4 benzoquinone triggers apoptosis in Jurkat cells. In this assay, Jurkat cells were incubated with either the red or blue 1,4 benzoquinone at a concentration of 25 μM for 0, 4, 8 and 12 h. Cells were then stained with the Fixable Viability Dye eFluor 780 and FITC Annexin V and analyzed using flow cytometry. Positive staining with Annexin V is a marker of early apoptosis, and positive staining with both Annexin V and the viability dye is a marker of late apoptosis/necrosis. Double negatives are live cells. Cells were grouped into three categories (live, early apoptotic, and late apoptotic/necrotic). Data from three independent experiments are shown as mean±SEM (standard error of mean).

FIG. 17 schematically illustrates the procedure of synthesizing the red benzoquinone:

FIG. 18 schematically illustrates the procedure of synthesizing the blue benzoquinone:

FIG. 19 illustrates the structures of the (Panel A) red and (Panel B) blue compounds extracted from the venom of Diplocentrus melici. Right panels show corresponding X-Ray crystallographic data (CCDC No. 0001001197099) of the synthetic molecules.

FIGS. 20A-20H illustrate the inhibitory activity (in vitro) of blue and red benzoquinones against Mycobacterium tuberculosis (H37Rv and MDR strain.

FIG. 20A is a graph illustrating minimal inhibitory concentrations (MICs) as determined by broth microdilution and bacterial proliferation evaluated by a colorimetric assay using Cell Titer 96® Aqueous. For both strains, the MIC of the blue benzoquinone against Mycobacterium tuberculosis was 4 μg/mL. The red benzoquinone had an MIC of 160 μg/mL.

FIG. 20B is a graph illustrating viability of the bacteria as evaluated by counting the colony-forming units resulting after treatment at the MIC values. Each result is mean±SD.

FIGS. 20C-20F are digital electron microscopy images showing ultrastructural changes in Mycobacterium tuberculosis in response to the blue benzoquinone.

FIG. 20C illustrates control untreated bacilli showed a well-defined, homogeneous and slightly electron-lucent cell wall, while the cytoplasm was generally electron lucent with some lipid medium-sized vacuoles.

FIG. 20D illustrates that after incubation with benzoquinone produced substantial abnormalities, such as extensive effacement of cell wall (arrow) and cytoplasmic extraction (asterisk).

FIGS. 20E and 20F illustrate conglomerates of electron dense reticular filaments located in the central areas of the cytoplasm.

FIGS. 20G and 20H illustrate similar subcellular changes induced by isoniazid incubation.

FIGS. 21A-21F illustrates inhibitory activity (in vivo) of the blue 1,4 benzoquinone against Mycobacterium tuberculosis. An experimental model of progressive pulmonary tuberculosis was used consisting of BALB/c mice infected with the Multi-Drug-Resistance (MDR) CIBIN99 strain. Mice were treated for two months with the blue 1,4 benzoquinone using a dose of 8 μg administered by intratracheal route every other day.

After the two months, the group of mice treated with the blue 1,4 benzoquinone had a marked improvement in their condition (FIG. 21A) with a reduction of more than 90% of the lung bacillary load (evaluated by counting the colony-forming units) compared to the untreated group (FIG. 21B). A reduction on the percentage of lung surface affected by pneumonia (LSAP) was observed in the lungs of the treated group. This difference was confirmed by automated histomorphometry that showed a 50% reduction of pneumonia post-treatment.

FIGS. 21C-21F illustrate representative micrographs of lung (hematoxylin-eosin staining, 250× magnification) from the untreated group (FIG. 21C) showing extensive areas of pneumonia (asterisk). There is less pneumonia in lungs of the treated group (FIG. 21D). A representative micrograph of a healthy mouse treated intratracheally for one month with 8 μg of the blue 1,4 benzoquinone is shown in FIG. 21E. The pulmonary histology is normal, with the exception of occasional mild inflammatory infiltrates found around venules (arrow). FIG. 21F shows that there is no fibrosis in the lungs of healthy control mice (trichrome Masson staining, magnification 200×).

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of molecular biology, microbiology, organic chemistry, biochemistry, physiology, cell biology, cancer biology, and the like, which are within the skill of the art. Such techniques are explained fully in the literature.

### Definitions

As used herein, “about,” “approximately,” and the like, when used in connection with a numerical variable, can generally refers to the value of the variable and to all values of the variable that are within the experimental error (e.g., within the 95% confidence interval for the mean) or within +/−10% of the indicated value, whichever is greater.

The term “administering” as used herein, can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrapulmonary intrathecal, intrahepatic, intralesional, and intracranial injections or infusion techniques. In some embodiments, the compounds and/or formulations thereof can be delivered directly to the lungs.

The term “agent” as used herein refers to any substance, compound, molecule, and the like, which can be biologically active or otherwise can induce a biological and/or physiological effect on a subject to which it is administered to. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

The term “alkoxy” as used herein refers to a linear or branched oxy-containing radical having an alkyl portion of one to about ten carbon atoms, such as a methoxy radical, which may be substituted. In aspects of the disclosure an alkoxy radical may comprise about 1-10, 1-8, 1-6 or 1-3 carbon atoms. In embodiments of the disclosure, an alkoxy radical comprises about 1-6 carbon atoms and includes a C1-C alkyl-O-radical wherein C1-C alkyl has the meaning set out herein. Examples of alkoxy radicals include without limitation methoxy, ethoxy, propoxy, butoxy, isopropoxy and tert-butoxy alkyls.

The term “antibiotic” refers to a compound or composition which decreases the viability of a microorganism, or which inhibits the growth or proliferation of a microorganism. The phrase “inhibits the growth or proliferation” means increasing the generation time (i.e., the time required for the bacterial cell to divide or for the population to double) by at least about 2-fold.

The term “anti-infective” as used herein refers to compounds or molecules that can either kill an infectious agent or inhibit it from spreading. Anti-infectives include, but are not limited to, antibiotics, antibacterials, antifungals, antivirals, and antiprotozoans.

The phrase “bacterial infection” as used herein refers to a bacteria colonizing a tissue or organ of a subject, where the colonization causes harm to the subject. The harm can be caused directly by the bacteria and/or by toxins produced by the bacteria. Reference to bacterial infection includes also includes bacterial disease. Antibiotic agents, such as those described herein, can kill bacteria, prevent bacterial growth, and/or assist the subject's ability to kill or prevent bacteria growth.

Bacteria that cause bacterial infection are called pathogenic bacteria. The terms “bacteria” or “bacterium” include, but are not limited to, Gram positive and Gram negative bacteria. Bacteria can include, but are not limited to, Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anaerobospirillum, Anabaena affinis and other cyanobacteria (including the Anabaena, Anabaenopsis, Aphanizomenon, Camesiphon, Cylindrospermopsis, Gloeobacter Hapalosiphon, Lyngbya, Microcystis, Nodularia, Nostoc, Phormidium, Planktothrix, Pseudoanabaena, Schizothrix, Spirulina, Trichodesmium, and Umezakia genera) Anaerorhabdus, Arachnia, Arcanobacterium, Arcobacter, Arthrobacter, Atopobium, Aureobacterium, Bacteroides, Balneatrix, Bartonella, Bergeyella, Bifidobacterium, Bilophila Branhamella, Borrelia, Bordetella, Brachyspira, Brevibacillus, Brevibacterium, Brevundimonas, Brucella, Burkholderia, Buttiauxella, Butyrivibrio, Calymmatobacterium, Campylobacter, Capnocytophaga, Cardiobacterium, Catonella, Cedecea, Cellulomonas, Centipeda, Chlamydia, Chlamydophila, Chromobacterium, Chyseobacterium, Chryseomonas, Citrobacter, Clostridium, Collinsella, Comamonas, Corynebacterium, Coxiella, Cryptobacterium, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Francisella, Fusobacterium, Gardnerella, Gemella, Globicatella, Gordona, Haemophilus, Hafnia, Helicobacter, Helococcus, Holdemania Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptotrichia, Leuconostoc, Listeria, Listonella, Megasphaera, Methylobacterium, Microbacterium, Micrococcus, Mitsuokella, Mobiluncus, Moellerella, Moraxella, Morganella, Mycobacterium, Mycoplasma, Myroides, Neisseria, Nocardia, Nocardiopsis, Ochrobactrum, Oeskovia, Oligella, Orientia, Paenibacillus, Pantoea, Parachlamydia, Pasteurella, Pediococcus, Peptococcus, Peptostreptococcus, Photobacterium, Photorhabdus, Phytoplasma, Plesiomonas, Porphyrimonas, Prevotella, Propionibacterium, Proteus, Providencia, Pseudomonas, Pseudonocardia, Pseudoramibacter, Psychrobacter, Rahnella, Ralstonia, Rhodococcus, Rickettsia Rochalimaea Roseomonas, Rothia, Ruminococcus, Salmonella, Selenomonas, Serpulina, Serratia, Shewenella, Shigella, Simkania, Slackia, Sphingobacterium, Sphingomonas, Spirillum, Spiroplasma, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Tropheryma, Tsakamurella, Turicella, Ureaplasma, Vagococcus, Veillonella, Vibrio, Weeksella, Wolinella, Xanthomonas, Xenorhabdus, Yersinia, and Yokenella. Other examples of bacterium include Mycobacterium tuberculosis, M. bovis, M. typhimurium, M. bovis strain BCG, BCG substrains, M. avium, M. intracellulare, M. africanum, M. kansasii, M. marinum, M. ulcerans, M. avium subspecies paratuberculosis, Staphylococcus aureus, Staphylococcus epidermidis, Staphylococcus equi, Streptococcus pyogenes, Streptococcus agalactiae, Listeria monocytogenes, Listeria ivanovii, Bacillus anthracis, B. subtilis, Nocardia asteroides, and other Nocardia species, Streptococcus viridans group, Peptococcus species, Peptostreptococcus species, Actinomyces israelii and other Actinomyces species, and Propionibacterium acnes, Clostridium tetani, Clostridium botulinum, other Clostridium species, Pseudomonas aeruginosa, other Pseudomonas species, Campylobacter species, Vibrio cholera, Ehrlichia species, Actinobacillus pleuro pneumoniae, Pasteurella haemolytica, Pasteurella multocida, other Pasteurella species, Legionella pneumophila, other Legionella species, Salmonella typhi, other Salmonella species, Shigella species, Brucella abortus, other Brucella species, Chlamydia trachomatis, Chlamydia psittaci, Coxiella burnetti, Escherichia coli, Neiserria meningitidis, Neiserria gonorrhea, Haemophilus influenzae, Haemophilus ducreyi, other Hemophilus species, Yersinia pestis, Yersinia enterolitica, other Yersinia species, Escherichia coli, E. hirae and other Escherichia species, as well as other Enterobacteria, Burkholderia cepacia, Burkholderia pseudomallei, Francisella tularensis, Bacteroides fragilis, Fudobascterium nucleatum, Provetella species, and Cowdria ruminantium, or any strain or variant thereof. Gram-positive bacteria may include, but are not limited to, Gram positive cocci (e.g., Streptococcus, Staphylococcus, and Enterococcus). Gram-negative bacteria may include, but are not limited to, Gram negative rods (e.g., Bacteroidaceae, Enterobacteriaceae, Vibrionaceae, Pasteurellae and Pseudomonadaceae).

The term “cancer”, as used herein, shall be given its ordinary meaning, as a general term for diseases in which abnormal cells divide without control. In particular, cancer refers to angiogenesis related cancer. Cancer cells can invade nearby tissues and can spread through the bloodstream and lymphatic system to other parts of the body.

There are several main types of cancer, for example, carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma is cancer that begins in the cells of the immune system.

When normal cells lose their ability to behave as a specified, controlled and coordinated unit, a tumor is formed. Generally, a solid tumor is an abnormal mass of tissue that usually does not contain cysts or liquid areas (some brain tumors do have cysts and central necrotic areas filled with liquid). A single tumor may even have different populations of cells within it, with differing processes that have gone awry. Solid tumors may be benign (not cancerous), or malignant (cancerous). Different types of solid tumors are named for the type of cells that form them. Examples of solid tumors are sarcomas, carcinomas, and lymphomas. Leukemias (cancers of the blood) generally do not form solid tumors.

Representative cancers include, but are not limited to, bladder cancer, breast cancer, colorectal cancer, endometrial cancer, head and neck cancer, leukemia, lung cancer, lymphoma, melanoma, non-small-cell lung cancer, ovarian cancer, prostate cancer, testicular cancer, uterine cancer, cervical cancer, thyroid cancer, gastric cancer, brain stem glioma, cerebellar astrocytoma, cerebral astrocytoma, glioblastoma, ependymoma, Ewing's sarcoma family of tumors, germ cell tumor, extracranial cancer, Hodgkin's disease leukemia, acute lymphoblastic leukemia, acute myeloid leukemia, liver cancer, medulloblastoma, neuroblastoma, brain tumors generally, non-Hodgkin's lymphoma, osteosarcoma, malignant fibrous histiocytoma of bone, retinoblastoma, rhabdomyosarcoma, soft tissue sarcomas generally, supratentorial primitive neuroectodermal and pineal tumors, visual pathway and hypothalamic glioma, Wilms' tumor, acute lymphocytic leukemia, adult acute myeloid leukemia, adult non-Hodgkin's lymphoma, chronic lymphocytic leukemia, chronic myeloid leukemia, esophageal cancer, hairy cell leukemia, kidney cancer, multiple myeloma, oral cancer, pancreatic cancer, primary central nervous system lymphoma, skin cancer, small-cell lung cancer, among others.

A tumor can be classified as malignant or benign. In both cases, there is an abnormal aggregation and proliferation of cells. In the case of a malignant tumor, these cells behave more aggressively, acquiring properties of increased invasiveness. Ultimately, the tumor cells may even gain the ability to break away from the microscopic environment in which they originated, spread to another area of the body (with a very different environment, not normally conducive to their growth), and continue their rapid growth and division in this new location. This is called metastasis. Once malignant cells have metastasized, achieving a cure is more difficult.

Benign tumors have less of a tendency to invade and are less likely to metastasize. Brain tumors spread extensively within the brain but do not usually metastasize outside the brain. Gliomas are very invasive inside the brain, even crossing hemispheres. They do divide in an uncontrolled manner, though. Depending on their location, they can be just as life threatening as malignant lesions. An example of this would be a benign tumor in the brain, which can grow and occupy space within the skull, leading to increased pressure on the brain.

The term “cell or population of cells” as used herein refers to an isolated cell or plurality of cells excised from a tissue or grown in vitro by tissue culture techniques. Most particularly, a population of cells refers to cells in vivo in a tissue of an animal or human. The term may also be applied to a population of bacteria either cultured in vitro or an infection in an animal or human subject.

The term “composition” as used herein refers to a product comprising the specified ingredients in the specified amounts, as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure and a pharmaceutically acceptable carrier.

The term “compound” as used herein refers in particular to 1,4 benzoquinones synthesized by oxidation precursor molecules in the venom of a scorpion species. The compounds of the disclosure can be prepared using reactions and methods generally known to the person of ordinary skill in the art and having regard to that knowledge and the disclosure of this application including the Examples. The reactions are performed in solvents appropriate to the reagents and materials used and suitable for the reactions being effected. It will be understood by those skilled in the art of organic synthesis that the functionality present on the compounds should be consistent with the proposed reaction steps. This may require modification of the order of the synthetic steps or selection of one particular process scheme over another in order to obtain a desired compound of the disclosure. It will also be recognized that another major consideration in the development of a synthetic route is the selection of any protecting group used for protection of the reactive functional groups present in the compounds described in this disclosure. An authoritative account describing the many alternatives to the skilled artisan is Greene and Wuts (Protective Groups In Organic Synthesis, Wiley and Sons, 1991).

A compound of the disclosure can contain one or more asymmetric centers and may give rise to enantiomers, diasteriomers, and other stereoisomeric forms which may be defined in terms of absolute stereochemistry as (R)- or (S)-. Thus, compounds of the disclosure include all possible diasteriomers and enantiomers as well as their racemic and optically pure forms. Optically active (R)- and (S)-isomers may be prepared using chiral synthons or chiral reagents, or resolved using conventional techniques. When a compound of the disclosure contains centers of geometric asymmetry, and unless specified otherwise, it is intended that the compounds include both E and A geometric isomers. All tautomeric forms are also included within the scope of a compound of the disclosure.

All chiral, diastereomeric, racemic forms of a structure are intended, unless a particular stereochemistry or isomeric form is specifically indicated. Compounds used in the present invention can include enriched or resolved optical isomers at any or all asymmetric atoms as are apparent from the depictions, at any degree of enrichment. Both racemic and diastereomeric mixtures, as well as the individual optical isomers can be isolated or synthesized so as to be substantially free of their enantiomeric or diastereomeric partners, and these are all within the scope of the invention.

As to any of the groups described herein, which contain one or more substituents, it is understood that such groups do not contain any substitution or substitution patterns which are sterically impractical and/or synthetically non-feasible. In addition, the compounds of this disclosed subject matter include all stereochemical isomers arising from the substitution of these compounds.

Selected substituents within the compounds described herein can be present to a recursive degree. In this context, “recursive substituent” means that a substituent may recite another instance of itself. Because of the recursive nature of such substituents, theoretically, a large number may be present in any given claim. One of ordinary skill in the art of medicinal chemistry and organic chemistry understands that the total number of such substituents is reasonably limited by the desired properties of the compound intended. Such properties include, by of example and not limitation, physical properties such as molecular weight, solubility or log P, application properties such as activity against the intended target, and practical properties such as ease of synthesis. Recursive substituents are an intended aspect of the disclosed subject matter. One of ordinary skill in the art of medicinal and organic chemistry understands the versatility of such substituents. To the degree that recursive substituents are present in a claim of the disclosed subject matter, the total number should be determined as set forth above.

When a group, e.g., an “alkyl” group, is referred to without any limitation on the number of atoms in the group, it is understood that the claim is definite and limited with respect the size of the alkyl group, both by definition; i.e., the size (the number of carbon atoms) possessed by a group such as an alkyl group is a finite number, less than the total number of carbon atoms in the universe and bounded by the understanding of the person of ordinary skill as to the size of the group as being reasonable for a molecular entity; and by functionality, i.e., the size of the group such as the alkyl group is bounded by the functional properties the group bestows on a molecule containing the group such as solubility in aqueous or organic liquid media. Therefore, a claim reciting an “alkyl” or other chemical group or moiety is definite and bounded, as the number of atoms in the group cannot be infinite.

The compounds of the invention and intermediates may be isolated from their reaction mixtures and purified by standard techniques such as filtration, liquid-liquid extraction, solid phase extraction, distillation, recrystallization or chromatography, including flash column chromatography, or HPLC.

The term “contacting a cell or population of cells” as used herein refers to delivering a compound or composition according to the present disclosure to an isolated or cultured cell or population of cells, bacteria or population of bacteria that are isolated, cultured or infecting an animal or human subject, or administering the compound in a suitable pharmaceutically acceptable carrier to the target tissue of an animal or human. Administration may be, but is not limited to, intravenous delivery, intraperitoneal delivery, intramuscularly, subcutaneously, or by any other method known in the art. One advantageous method is to deliver directly into a blood vessel leading into an infected or cancerous tissue or organ.

The term “derivative” as used herein refers to any compound having the same or a similar core structure to the compound but having at least one structural difference, including substituting, deleting, and/or adding one or more atoms or functional groups. The term “derivative” does not mean that the derivative is synthesized from the parent compound either as a starting material or intermediate, although this may be the case. The term “derivative” can include prodrugs, or metabolites of the parent compound. Derivatives can include the oxidized products of parent compounds. As used herein, “dose,” “unit dose,” or “dosage” can refer to physically discrete units suitable for use in a subject, each unit containing a predetermined quantity of a compound described herein and/or a pharmaceutical formulation thereof calculated to produce the desired response or responses in association with its administration.

The term “effective amount” as used herein refers to the amount of a compound provided herein that is sufficient to effect beneficial or desired biological, emotional, medical, or clinical response of a cell, tissue, system, animal, or human. An effective amount can be administered in one or more administrations, applications, or dosages. The term cam also include within its scope amounts effective to enhance or restore to substantially normal physiological function. The “effective amount” can refer to the amount of a compound described herein (e.g. the 1,4 benzoquinones and/or derivatives thereof) that can kill or inhibit bacteria.

The term “pharmaceutically acceptable carrier” as used herein refers to a diluent, adjuvant, excipient, or vehicle with which a probe of the disclosure is administered and which is approved by a regulatory agency of the Federal or a state government or listed in the U.S. Pharmacopeia or other generally recognized pharmacopeia for use in animals, and more particularly in humans. Such pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. When administered to a patient, the probe and pharmaceutically acceptable carriers can be sterile. Water is a useful carrier when the probe is administered intravenously. Saline solutions and aqueous dextrose and glycerol solutions can also be employed as liquid carriers, particularly for injectable solutions. Suitable pharmaceutical carriers also include excipients such as glucose, lactose, sucrose, glycerol monostearate, sodium chloride, glycerol, propylene, glycol, water, ethanol and the like. The present compositions, if desired, can also contain minor amounts of wetting or emulsifying agents, or pH buffering agents. The present compositions advantageously may take the form of solutions, emulsion, sustained-release formulations, or any other form suitable for use.

The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically functional derivative” as used herein refers to any pharmaceutically acceptable derivative of a compound of the disclosure, for example, an ester or an amide, which upon administration to a subject is capable of providing (directly or indirectly) a compound of the disclosure or an active metabolite or residue thereof. Such derivatives are recognizable to those skilled in the art, without undue experimentation (see for example Burger's Medicinal Chemistry and Drug Discovery, 5.sup.th Edition, Vol 1: Principles and Practice, which has illustrative pharmaceutically functional derivatives).

The terms “subject”, “individual”, or “patient” as used herein are used interchangeably and refer to an animal preferably a warm-blooded animal such as a mammal. Mammal includes without limitation any members of the Mammalia. A mammal, as a subject or patient in the present disclosure, can be from the family of Primates, Carnivora, Proboscidea, Perissodactyla, Artiodactyla, Rodentia, and Lagomorpha. In a particular embodiment, the mammal is a human. In other embodiments, animals can be treated; the animals can be vertebrates, including both birds and mammals. In aspects of the disclosure, the terms include domestic animals bred for food or as pets, including equines, bovines, sheep, poultry, fish, porcines, canines, felines, and zoo animals, goats, apes (e.g. gorilla or chimpanzee), and rodents such as rats and mice.

The term “substantially pure” as used herein, can mean an object species is the predominant species present (i.e., on a molar basis it is more abundant than any other individual species in the composition), and preferably a substantially purified fraction is a composition wherein the object species comprises about 50 percent of all species present. Generally, a substantially pure composition will comprise more than about 80 percent of all species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single species.

The terms “sufficient” and “effective” as used herein refers to an amount (e.g. mass, volume, dosage, concentration, and/or time period) needed to achieve one or more desired result(s). For example, a therapeutically effective amount refers to an amount needed to achieve one or more therapeutic effects.

The term “therapeutic effect” as used herein refers to an effect of a composition of the disclosure, in particular a formulation or dosage form, or method disclosed herein. A therapeutic effect may be a sustained therapeutic effect that correlates with a continuous concentration of a compound of the disclosure over a dosing period, in particular a sustained dosing period. A therapeutic effect may be a statistically significant effect in terms of statistical analysis of an effect of a compound of the disclosure versus the effects without the compound.

The term “therapeutically effective amount” relates to the amount or dose of an active compound of the disclosure or composition comprising the same that will lead to one or more desired effects, in particular, one or more therapeutic effects or beneficial pharmacokinetic profiles. A therapeutically effective amount of a substance can vary according to factors such as the disease state, age, sex, and weight of the subject, and the ability of the substance to elicit a desired response in the subject. A dosage regimen may be adjusted to provide the optimum therapeutic response or pharmacokinetic profile. For example, several divided doses may be administered daily or the dose may be proportionally reduced as indicated by the exigencies of the therapeutic situation.

The term “treating” and “treatment” can refer generally to obtaining a desired pharmacological and/or physiological effect. The effect can be, but does not necessarily have to be, prophylactic in terms of preventing or partially preventing a disease, symptom or condition thereof, such as a bacterial infection. The effect can be therapeutic in terms of a partial or complete cure of a disease, condition, symptom or adverse effect attributed to the disease, disorder, or condition. The term “treatment” as used herein covers any treatment of a bacterial infection (including but not limited to a Staphylococcus species infection (such as but not limited to a Staphylococcus aureus infection) in a subject, particularly a human, and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting a disease, disorder or condition such as a cancer, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

The term “unit dosage form” as used herein refers to physically discrete units suitable as unitary dosages for human patients and other mammals with each unit containing a predetermined quantity of active material calculated to produce the desired therapeutic effect in association with suitable pharmaceutical carriers or excipients. The compositions according to the present disclosure may be formulated in a unit dosage form. A single daily unit dose also may be divided into 2 or 3 unit doses that are taken at different times throughout the day, or as a controlled release form, so as to reduce adverse side-effects as much as possible.

### Abbreviations

MDR, multi-drug resistant; RP-HPLC, reverse-phase HPLC; MIC, minimal inhibitory concentration; CID-MS/MS, collision induced dissociation tandem mass spectrometry; HMBC, Heteronuclear Multiple Bond Correlation PBMC, peripheral blood mononuclear cell; DTNB; 5,5′-dithiobis-(2-nitrobenzoic acid); DCFH-DA dichloro-dihydro-fluorescein diacetate; SEM, standard error of mean; i.v., intravenously; i.m., intramuscularly; s.c., subcutaneously; i.d., intradermally; ROS, reactive oxygen species; HDX, Hydrogen-deuterium exchange; NOE, nuclear Overhauser effect; CAN, ceric ammonium nitrate; CFU, colony-forming units; TFA, trifluoroacetic acid.

### DISCUSSION

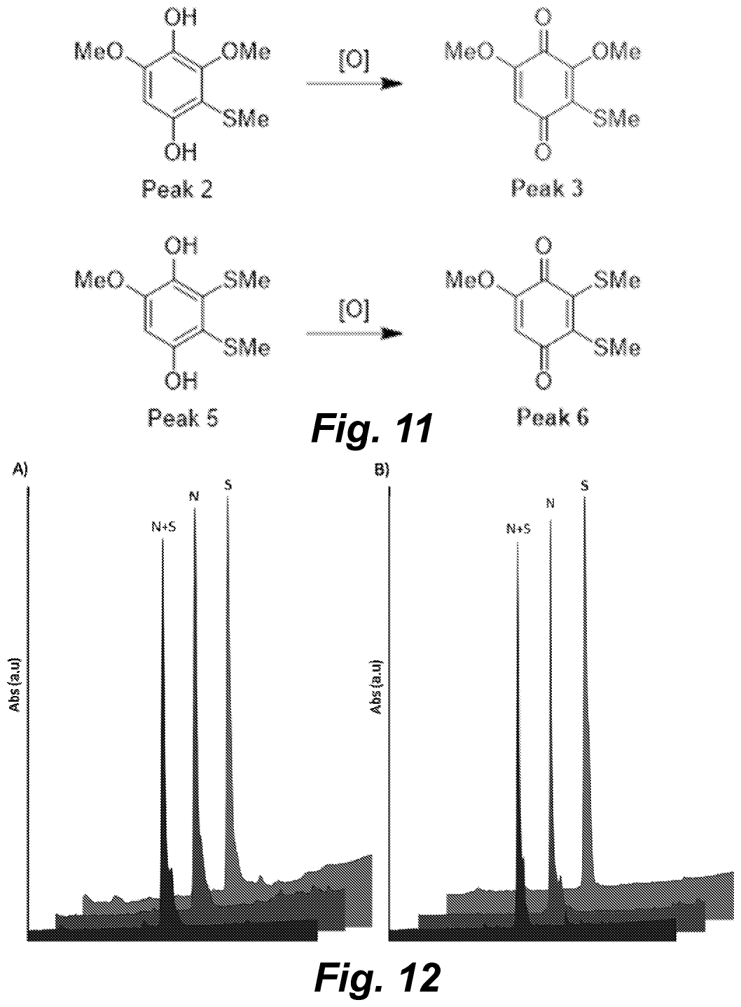

Compounds with the 1,4-benzoquinone motif are a large class of molecules that are highly reactive, acting as both oxidants and Michael acceptors, and many have been serendipitously found to have antimicrobial, antineoplastic, anticoagulant, and analgesic activity (Finley K. T. (2010) Quinonoid Compounds (1974); Brunmark & Cadenas (1988) Chemico-Biol. Interacts 68: 273-298; Abraham et al., (2011) J. Brazilian Chem. Soc. 22: 385-421; Novais et al. (2017) RSC Advances 7: 18311-18320; Lana et al., (2006) J. Agricult. Food Chem. 54: 2053-2056; Schulz et al. (2011) J. Antibiotics 64: 763-768; Zhang et al. (2016) Chem. Pharm. Bull. 64: 1036-1042). The two 1,4-benzoquinone compounds of the disclosure, one red, the other blue, which are derived from naturally occurring precursors in the venom of Diplocentrus melici, a scarcely studied species of scorpion indigenous to Mexico. The naturally occurring precursors rapidly oxidize upon exposure to air. Although their identities could not be conclusively determined, it is probable that they are the corresponding hydroquinones (FIG. 11) (Hassan et al., (2017) J. Am. Soc. Mass Spect. 28: 270-277). It is currently unknown why the scorpion telson contains such oxidatively labile compounds in great abundance. Since they are not in contact with O₂ (air) prior to injection into a target tissue, it is possible that their function in nature requires the unoxidized state.

To gain access to sufficient quantities of both 1,4 benzoquinones for biological testing, synthetic routes from commercially available reagents were used. The resulting synthetic 1,4 benzoquinones have the same structure, biological activity, and physicochemical properties as the non-natural oxidized compounds isolated from air-exposed precursors in the extracted venom. These colored 1,4 benzoquinones are advantageous as lead compounds for the development of antimicrobial agents against S. aureus and M. tuberculosis.

The 1,4 benzoquinones of the present disclosure, or a pharmaceutically acceptable salt thereof (or a pharmaceutical compositions containing 1,4 benzoquinones of the present disclosure or a pharmaceutically acceptable salt thereof), can be administered to a patient by any route that results in prevention or alleviation of symptoms associated with the particular neurological condition. For example, as described in more detail below, 1,4 benzoquinones of the present disclosure or a pharmaceutically acceptable salt thereof can be administered parenterally, intravenously (I.V.), intramuscularly (I.M.), subcutaneously (S.C.), intradermally (I.D.), orally, intranasally, etc. Examples of intranasal administration can be by means of a spray, drops, powder or gel. However, other means of drug administrations are well within the scope of the present invention.

1,4 benzoquinones of the present disclosure may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

Against S. aureus, both compounds are highly active, with potencies comparable to commercially used antibiotics. The red 1,4 benzoquinone is slightly more active than the blue one (MIC of 4 μg/mL vs. MIC of 6 μg/mL). The inhibitory activity of both compounds is greater than that of naturally occurring benzoquinones known to inhibit the growth of S. aureus (Hassan et al., (2017) J. Am. Soc. Mass Spect. 28: 270-277; Kim et al. (2010) J. Microbiol. Biotech. 20: 1204-1209). Despite such dramatic activity against S. aureus (Gram-positive), neither compound was found to have appreciable activity against Gram-negative E. coli. Previous studies have delineated that substitution at the 3-position of 2,6-dimethoxybenzoquinone dramatically decreases its activity against E. coli (Lana et al., (2006) J. Agricult. Food Chem. 54: 2053-2056). It is possible that the thiomethoxy group is responsible for the selectivity seen in our compounds.

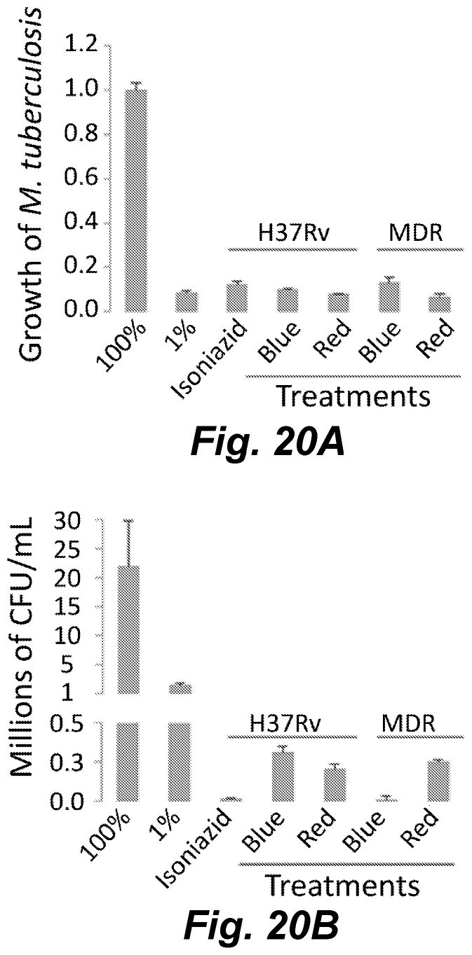

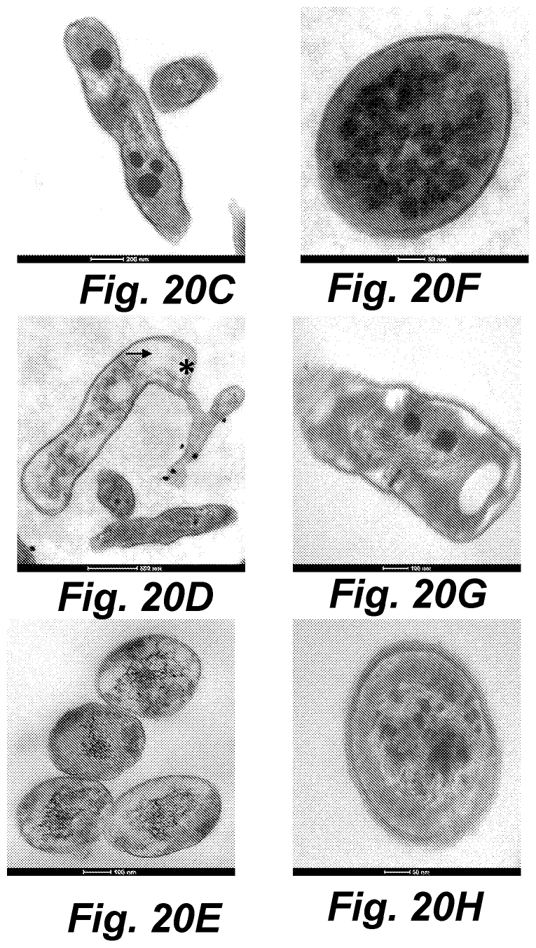

In addition to the antimicrobial activity against S. aureus, both 1,4 benzoquinones inhibit the growth of M. tuberculosis, with the blue 1,4 benzoquinone being most advantageous promising for a clinical application (MIC of 4 μg/mL) (FIG. 20A). However, there was a reduction in the bacterial concentration after the treatment for both 1,4 benzoquinones in comparison to the initial inoculum concentration of Mycobacteria (2.5×10⁶ bacteria/mL) suggesting bactericidal activity. This in vitro bactericidal effect is higher in the MDR strain with the blue 1,4 benzoquinone killing more than 90% of the bacteria load at the MIC (FIG. 20B). This bactericidal effect is also evident in an in vivo experimental infection mouse model; treated mice have a diminution of the bacillary load and a clear decrease in tissue damage. The MIC of the blue 1,4 benzoquinone (4 μg/mL) is markedly lower than related compounds that appear in the literature, including lapachol (MIC=100 μg/mL), 1,4-napthoquinone (MIC=100 μg/mL), and 1,4-benzoquinone (MIC=25 μg/mL) (Tran et al. (2004) Bioorganic Med. Chem. 12: 4809-4813).

The high reactive oxygen species (ROS) production produced by benzoquinones in eukaryotic cells could also be the mechanism of the anti-mycobacterial activity. Several bactericidal antibiotics with a variety of different mechanisms of action increase ROS production within cells via the Fenton reaction (Kohanski et al., (2007) Cell 130: 797-810; Wang & Zhao (2009) Antimicrob. Agents Chemotherapy 53: 1395-1402). Numerous ROS, in particular hydroxyl radicals, induce bacterial death via DNA damage, which is caused in part by the oxidation of the guanine nucleotide pool (Foti et al., (2012) Science 336: 315-319). The remarkable activity against the MDR strain proves the potential of the blue 1,4 benzoquinone as a lead molecule for treatment of infection caused by multi-drug resistant M. tuberculosis.

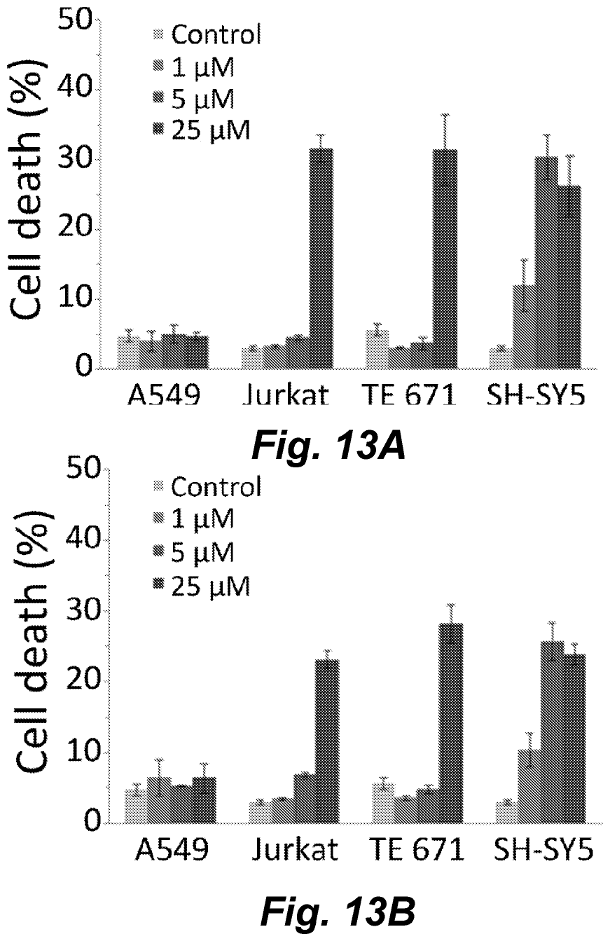

The cytotoxicity of these compounds to T-cell leukemia, rhabdomyosarcoma, and metastatic neuroblastoma but not in the lung adenocarcinoma cell line (FIGS. 13A and 13B) suggests a selectivity for inhibitory activity against some cell lines. The 1,4-benzoquinone compounds are oxidatively very labile; they are easily reduced to semiquinones which are rapidly re-oxidized by molecular oxygen. This vigorous redox activity generates a multitude of reactive oxygen species (ROS) as byproducts, including peroxides, superoxides, and hydroxyl radicals (Saibu et al., (2014) Anticancer Res. 34: 4077-4086; Gutierrez P. L. (2000) Frontiers Bioscience 5: D629-638). By oxidizing thiol groups into disulfides, these ROS deplete intracellular glutathione and cause protein aggregation and malfunction (Mytilineou et al., (2002) Parkinsonism Relat. Disord. 8: 385-387; Wilhelm et al., (1997) Mol. Cellular Biol. 17: 4792-4800). ROS irreversibly damage other biological macromolecules essential to cell survival, including lipids and nucleic acids (Wang et al. (2006) Proc. Nat. Acad. Sci. U.S.A. 103: 3604-3609; Green & Reed (1998) Science 281: 1309-1312). Once cellular damage reaches a threshold, apoptotic pathways are initiated for systematic cell death. Such is the mechanism of action of the naturally occurring cytotoxic quinones doxorubicin, daunorubicin, and mitomycin C, which are clinically used chemotherapeutic agents (Saibu et al., (2014) Anticancer Res. 34: 4077-4086).

In this matter, the apoptotic activity mediated by ROS could enhance the antimicrobial activity against intracellular M. tuberculosis, because it is known that this pathogen inhibits apoptosis of infected macrophages (Briken & Miller (2008) Future Microbiol. 3: 415-422; Lam et al. (2017) Am. J. Physiol.-Lung Cell. Mol. Physiol. 313:L218-L229). Induction of apoptosis plus the direct bactericidal activity of the blue 1,4 benzoquinone could have a synergistic effect in the eradication of tuberculosis in vivo.

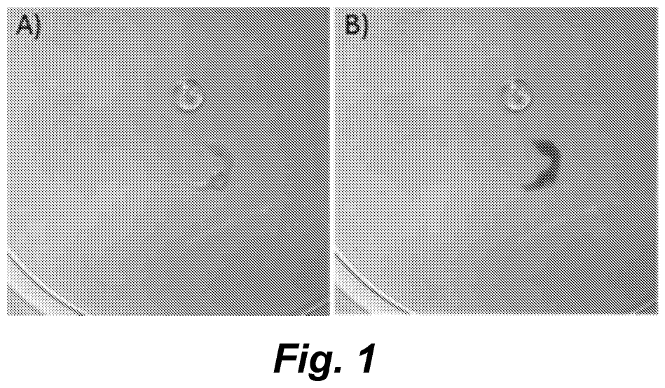

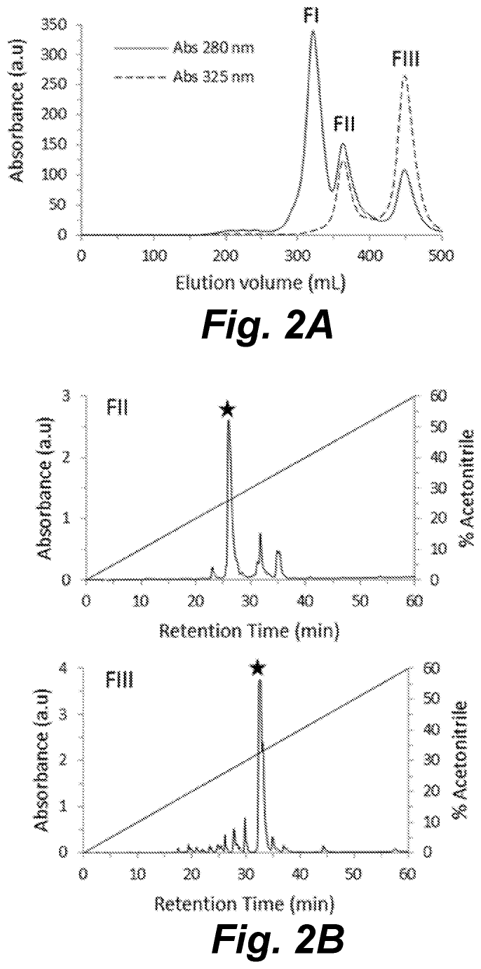

Isolation and purification of the red and blue compounds from the venom The total venom extracted by electrical stimulation from the telson of Diplocentrus melici. The extracted venom was first exposed to air until its color changed from colorless to dark red (FIG. 1). This red-colored viscous liquid was dissolved in ammonium acetate (20 mM, pH 4.7) and separated by gel filtration by a Sephadex G-50 column into three major fractions (labelled FI, FII, FIII in FIG. 2A). FI was colorless and had a strong absorption at 280 nm, indicating a composition largely of peptides, as shown by previous studies on scorpion venom (Possani et al., (2000) Biochimie 82: 861-868). In contrast, fractions FII and FIII were red and had strong absorbances at both 280 nm and 325 nm. When lyophilized, fractions FII and FIII yielded a red and blue powder, respectively.

FII and FIII were further purified by reversed-phase HPLC (C18 column, 0 to 60% acetonitrile in water, 60 min) as shown in FIG. 2B. An intense peak at 25.2 min elution time in the chromatogram corresponded to a single red compound (red in solution and in the dried state), and another at 32.7 min corresponded to a single blue compound (red in solution but blue in the dried state). The purified compounds were collected and dried for structural characterization and evaluation of biological activities.

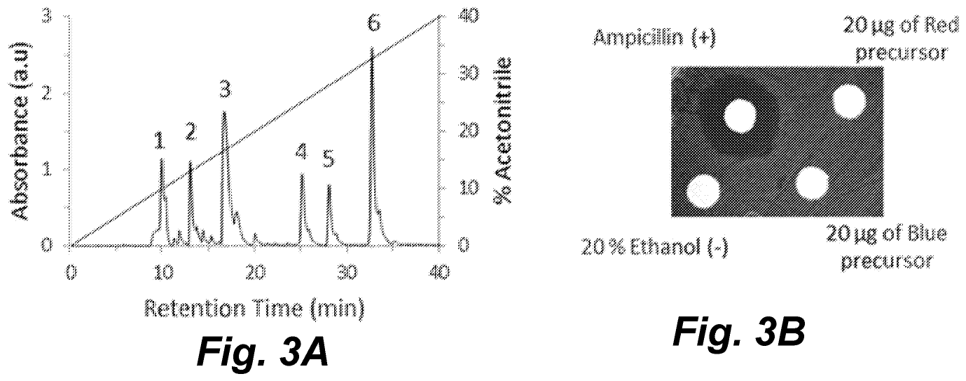

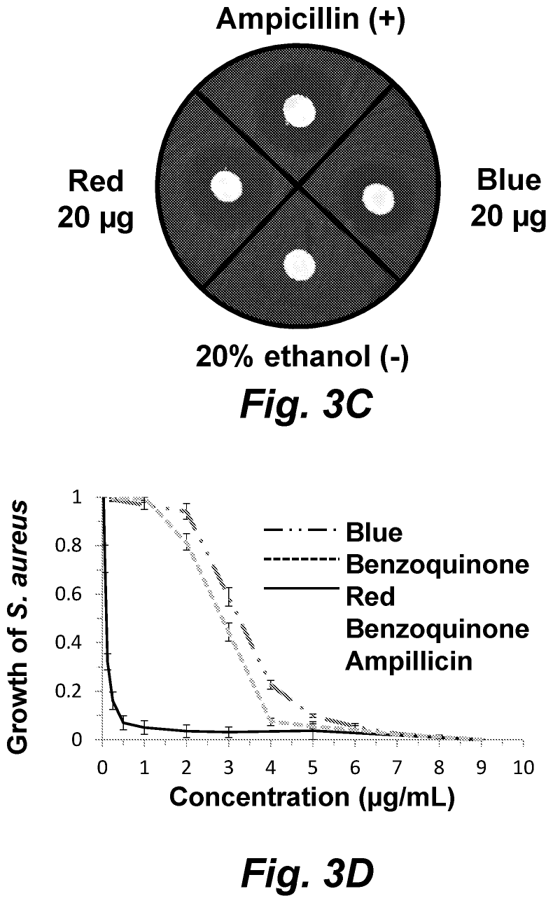

The colored compounds were formed by oxidation of colorless native precursor compounds present in the Diplocentrus melici venom that had not been exposed to air (O₂) in the venom or in the telson of the scorpion. This transformation started within the first seconds of exposure to air (FIG. 1, left panel). Within ten minutes, the entire liquid extracted from the scorpions reddened (FIG. 1, right panel). To isolate the precursors and identify these colored compounds, an alternative purification protocol was performed. The rate of oxidation of the red and blue compounds was markedly reduced when the venom immediately dissolved in acetone with minimal exposure to air. Rapid fractionation of this acetonic solution by HPLC yielded 6 main peaks (FIG. 3A). Peaks 4 and 6 can be attributed to the red and the blue compounds, respectively.

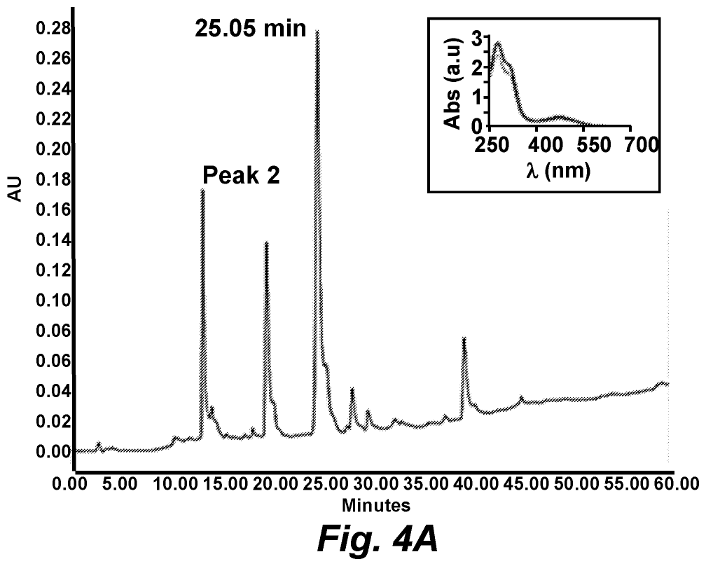

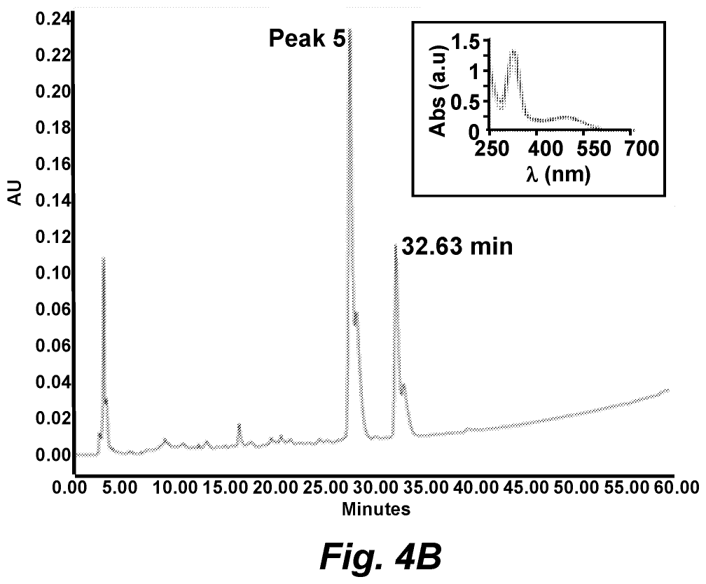

The fractions corresponding to the peaks were recovered and bubbled with air in order to promote oxidation with dissolved oxygen. After treatment, the compound in peak 2 underwent a shift in the retention time from 13.06 to 25.05 min (FIG. 4A). Similarly, the compound in peak 5 underwent a shift in retention time from 28.09 to 32.63 min (FIG. 4B). After prolonged exposure to air, the compounds in these fractions had the same UV-vis absorbance spectra as the red and blue compounds formed by exposing the extracted venom to air, respectively. Accordingly, the compounds contained in peaks 4 and 6 prior to oxidation are precursors of the red and blue compounds synthesized by exposure to air. Fractions corresponding to Peaks 1 and 3 did not change color upon exposure to air; at present, the composition of these peaks is unknown. The precursor components 2 and 5 (in their unoxidized states) were assayed for possible inhibitory effect on Staphylococcus aureus using a disk-diffusion assay. No inhibition was observed as shown in FIG. 3B. There was no appearance of color on the disks or in the agar during the incubation time.

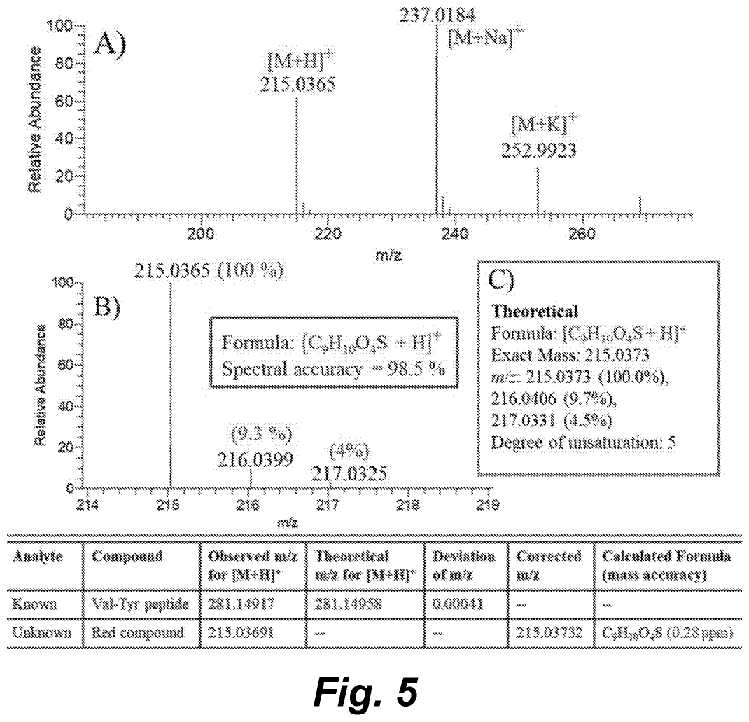

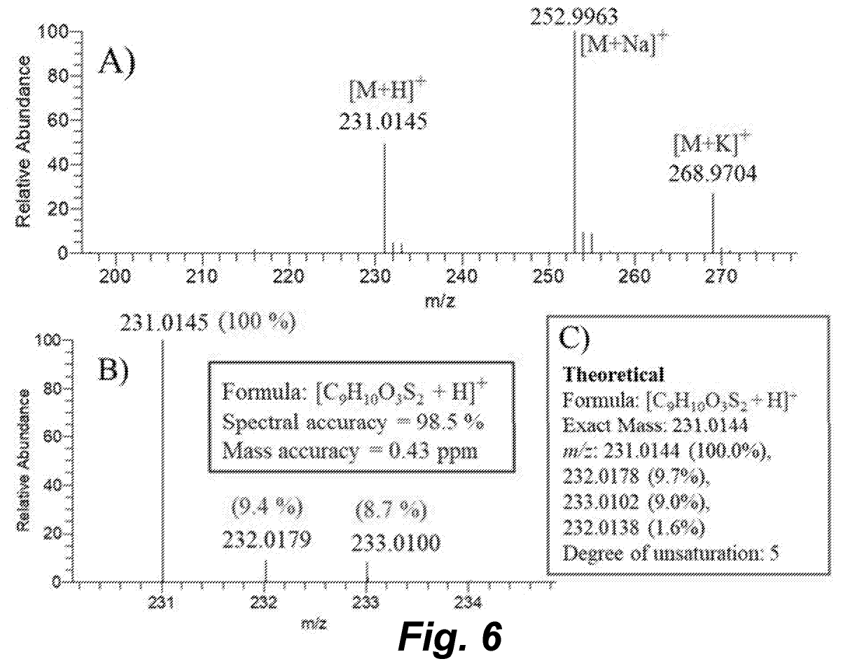

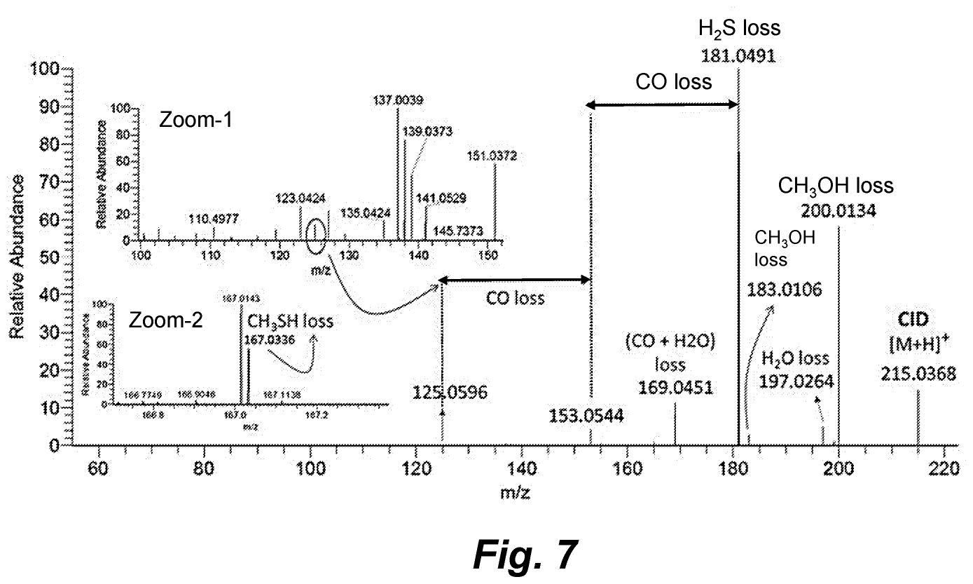

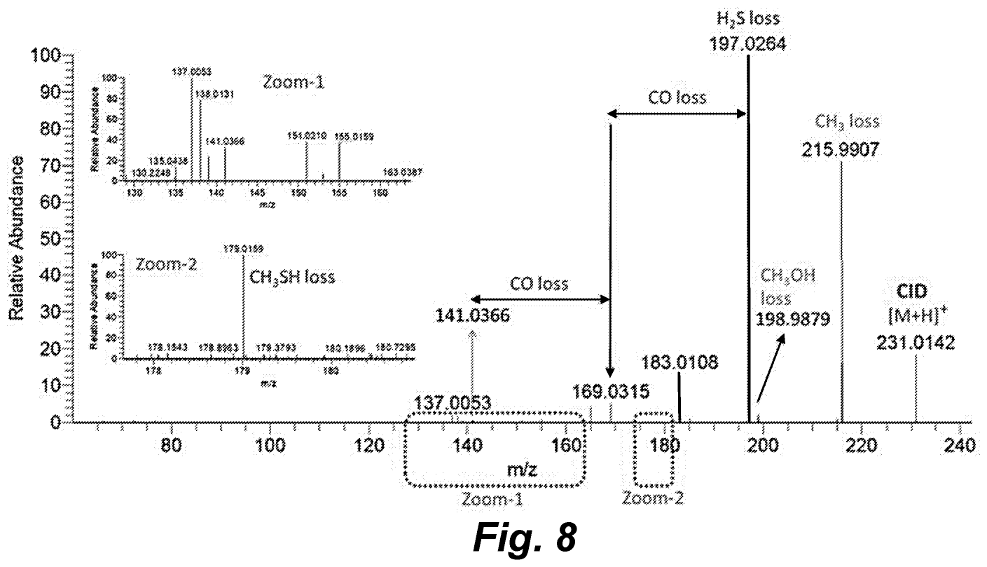

Structural characterization of the red and blue compounds The red and blue compounds were electrosprayed from a methanolic solution, which produced ion signals of both protonated and metallated species (FIGS. 5 and 6). Both high mass accuracy and isotope distribution data suggested the molecular formulas C₉H₁₀O₄S and C₉H₁₀O₃S₂ for the red and blue compounds, respectively, indicating in each case five degrees of unsaturation (number of rings and double bonds), calculated as one-half the sum of (twice the number of carbon atoms plus 2 minus the number of hydrogen atoms). The experimental mass-to-charge (m/z) accuracy was 0.28 ppm for the red compound and 0.43 ppm for the blue compound (FIGS. 5 and 6). Hydrogen-deuterium exchange (HDX) experiments with each compound did not show evidence indicating the presence of exchangeable hydrogens (e.g., —OH, —SH, etc.) in each molecule. The tandem mass spectrometric study (FIGS. 7 and 8) using collision induced dissociation (CID) on the protonated species (m/z 215.0365) for the red compound and the protonated species (m/z 231.0145) for the blue compound indicated the presence of carbonyl (C═O), methylthio (—S-Me), and methoxy (—O-Me) functional groups in the structure of each compound. The ¹H NMR spectrum strongly suggested the presence of one —S-Me functional group (δH 2.55, s, 3H) and two —O-Me functional groups [(δH 3.81, s, 3H) and (δH 3.95, s, 3H)] in the red compound. For the blue compound, the same experiment strongly suggests the presence of two —S-Me functional groups [(δH 2.64, s, 3H) and (δH 2.52, s, 3H)] and one —O-Me functional group (δH 3.81, s, 3H). A singlet signal of one proton at δH 5.96 for the red compound and δH 6.01 for the blue compound was also detected, suggesting the presence of a vinylic proton in each molecule.

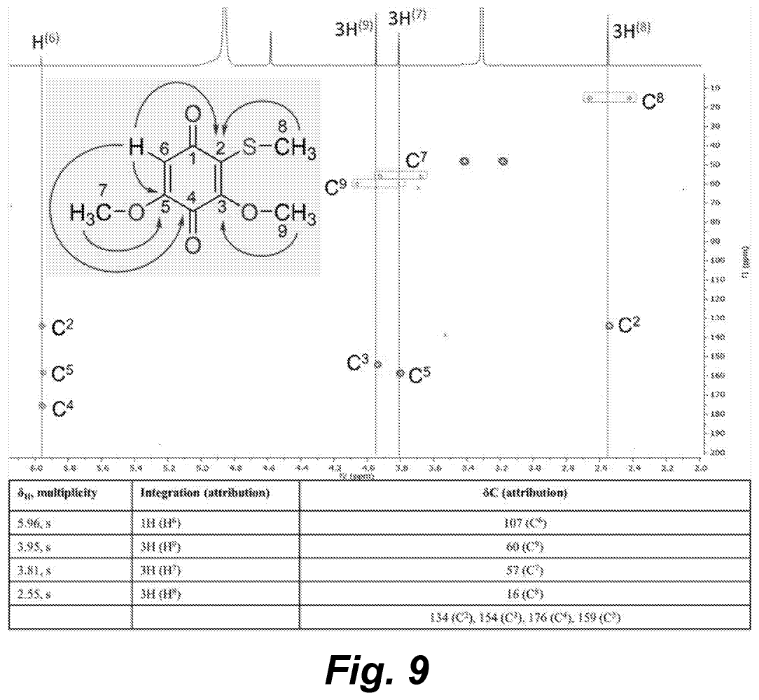

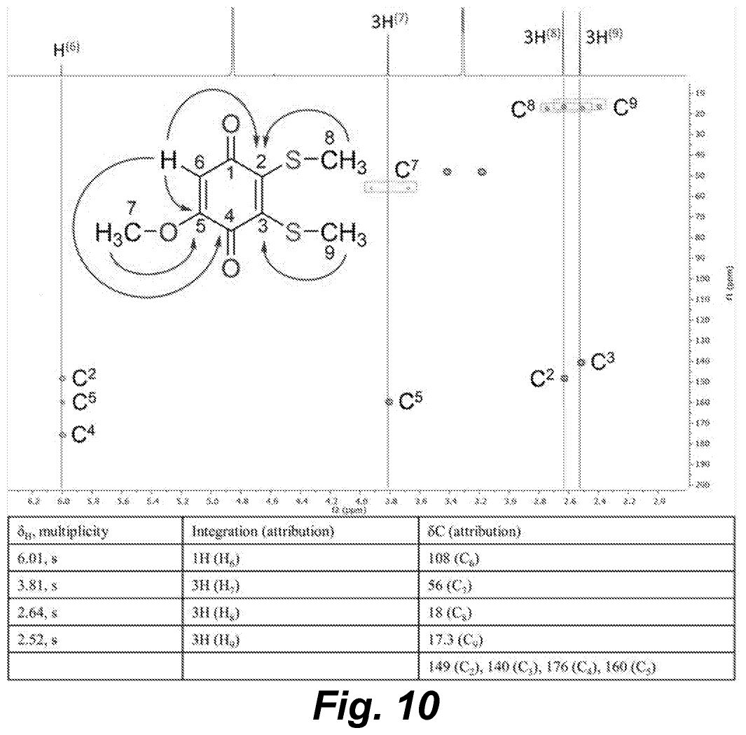

¹H-¹H COSY correlation was not seen in either molecule, suggesting the lack of spin-spin coupling between neighboring protons. Individual carbon chemical shifts from HMBC and HSQC experiments (vide infra) were recorded Because of low sample amounts, the HMBC experiment was conducted in a Shigemi advanced NMR tube (solvent: Methanol-d4). The results are shown in FIGS. 9 and 10. Detailed analysis of carbon-hydrogen correlation in HMBC revealed that the red and blue compounds are quinone derivatives, 3,5-dimethoxy-2-(methylthio)cyclohexa-2,5-diene-1,4-dione and 5-methoxy-2,3-bis(methylthio)cyclohexa-2,5-diene-1,4-dione, respectively, whose structures are given in FIG. 9. FIGS. 9 and 10 also list the individual chemical shifts of protons and carbons. The NOE (nuclear Overhauser effect) study also revealed the proximity of a vinylic proton (δH 5.96) to the —O-Me functional group (δH 3.81) in the red compound and a vinylic proton (δH 6.01) to the —O-Me functional group (δH 3.81) in the blue compound. As the structures of the red and blue compounds have been established as 1,4-benzoquinones, the molecules corresponding to peaks 2 and 5 (FIG. 3A) are likely to be precursor hydroquinones, which were oxidized in air to form 1,4-benzoquinones as discussed before (FIG. 11). Each 1,4 benzoquinone derivative was synthesized and the structure of each synthesized compound was further verified by comparing their NMR data with wild-type compounds.

### Chemical Synthesis of the Red and Blue Benzoquinones

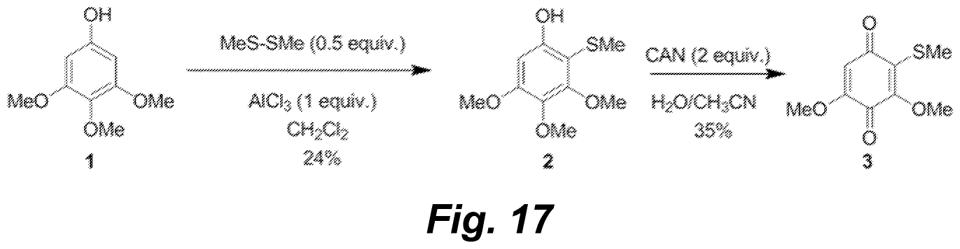

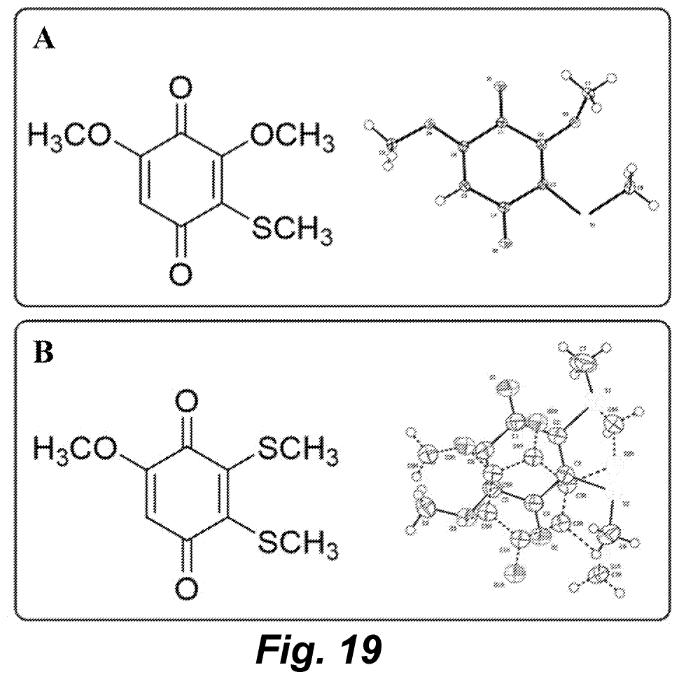

The red compound was synthesized in a two-step procedure as shown in Scheme S1 (FIG. 17). 3,4,5-trimethoxyphenol 1 was reacted with dimethyl disulfide in the presence of aluminum chloride in a Friedel-Crafts type reaction to form 3,4,5-trimethoxy-2-(methylthio)phenol 2 with moderate yield of 24%. This intermediate was oxidized with ceric ammonium nitrate (CAN) and recrystallized from 1:4 EtOAc/hexanes to yield (35%) red crystals of the desired 1,4 benzoquinone compound 3 (X-ray crystal structure shown in FIG. 19, top right panel).

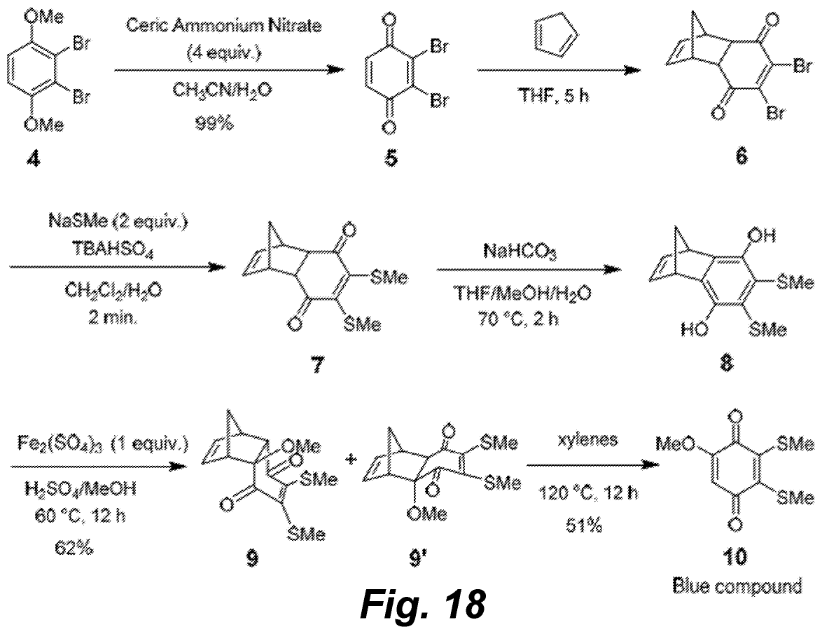

Scheme S2 (FIG. 18) illustrates the synthesis of the blue compound. 1,4-dimethoxy-2,3-dibromobenzene 4 was oxidized with CAN to provide 2,3-dibromo-1,4-benzoquinone 5 in an almost quantitative reaction. A Diels-Alder cycloaddition between 5 and excess, freshly distilled cyclopentadiene proceeded smoothly at room temperature, yielding tricyclic compound 6. Using an optimized protocol (Ferreira et al., (2003) Tetrahedron 59: 1349-1357) incorporated herein by reference in its entirety, the bromides were replaced with thiomethoxy groups in a fast reaction conducted in a separatory funnel. Heating 7 with an excess of NaHCO₃ in a 1:1:1 mixture of THF/MeOH/H₂O gave the enol tautomer 8. Treatment of 8 with Fe₂(SO₄)₃ in acidic MeOH at 60° C. for 12 h lead to rapid oxidation to the 1,4-benzoquinone; compounds 9 and 9′ formed via a 1,4-addition of MeOH to this benzoquinone. A retro-Diels-Alder reaction was effected at 120° C. to yield the blue 1,4 benzoquinone 10 (X-ray crystal structure shown in FIG. 19).

### Biological Activity of the Red and Blue Benzoquinones

The inhibitory activity of the red and blue 1,4 benzoquinones against Staphylococcus aureus was evaluated using a disk-diffusion assay. There was a clear inhibition of S. aureus growth by the action of the red and blue 1,4 benzoquinones (FIG. 3C). Based on the diameters of inhibition, the red 1,4 benzoquinone is more active than the blue 1,4 benzoquinone. This difference was confirmed using a broth microdilution assay (FIG. 3D). The minimum inhibitory concentration (MIC) for S. aureus growth was 4 μg/mL for the red 1,4 benzoquinone and 6 μg/mL for the blue 1,4 benzoquinone (FIG. 3D, Table 1). Ampicillin (MIC=0.5 μg/mL) was used as a positive control (ESCMID Eo (2003) Clin. Microbiol. Infect. 9: ix-xv; Pieterse et al., (2010) Brazil. J. Microbiol. 41: 133-145). These compounds were bactericidal at their MICs, killing 90% of S. aureus in 6 h and 99.9% in 24 h (Table 2). Neither 1,4 benzoquinone showed activity against gram negative E. coli and the pathogenic fungus Candida. albicans.

Using a broth microdilution assay, the efficacy of both 1,4 benzoquinones in killing Mycobacterium tuberculosis H37Rv (a pathogenic strain commonly used in such studies (Bifani et al. (2000) J. Clin. Microbiol. 38: 3200-3204)) and a multi-drug resistant (MDR) strain from clinical isolates was tested. Only the blue 1,4 benzoquinone showed significant inhibitory activity against Mycobacterium tuberculosis (H37Rv and MDR) with an MIC of 4 μg/mL for both strains (FIG. 20A), which is similar to the MICs reported for isoniazid, rifampicin, ethambutol, levofloxacin, moxifloxacin, and capreomycin against sensitive strains of M. tuberculosis (Chanwong et al., (2007) Tuberculosis 87: 130-133; Kaniga et al. (2016) J. Clin. Microbiol 54: 2963-2968; SturegArd et al. (2015) Clin. Microbiol. Infect. 21: 148.e145-148.e147). The red benzoquinone was inhibitory only at concentrations higher than 160 μg/mL.

In an independent test of efficacy, the concentration of colony-forming units (CFU) of bacteria growing after treatment was determined. There was a reduction in the concentration of CFUs after treatment with the 1,4 benzoquinones at doses equivalent to their MICs (FIG. 20B), especially by the blue 1,4 benzoquinone in the MDR strain with more than 90% of killing in comparison to the inoculum bacteria concentration. Additionally, ultrastructural changes were promoted by the action of the blue 1,4 benzoquinone in the M. tuberculosis bacilli (FIGS. 20C-20F). These were similar to what occurs when the bacteria are exposed to isoniazid (FIG. 20G-20H), an effective anti-mycobacterial antibiotic that interferes with cell wall synthesis. There was a loss of the elongated bacillus cytomorphology as well as the formation of cytoplasmic electro-dense conglomerates with extensive cell-wall effacement.

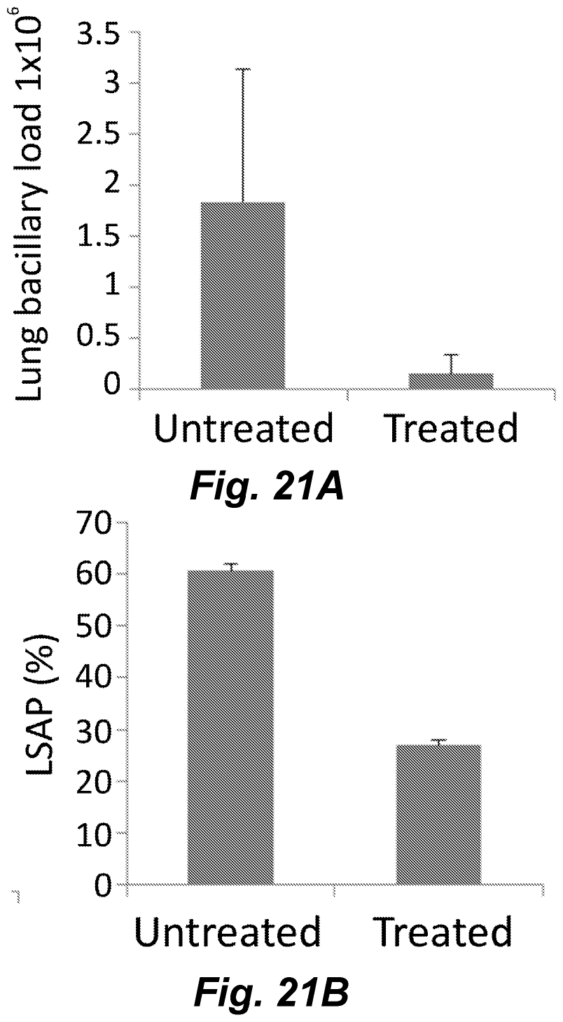

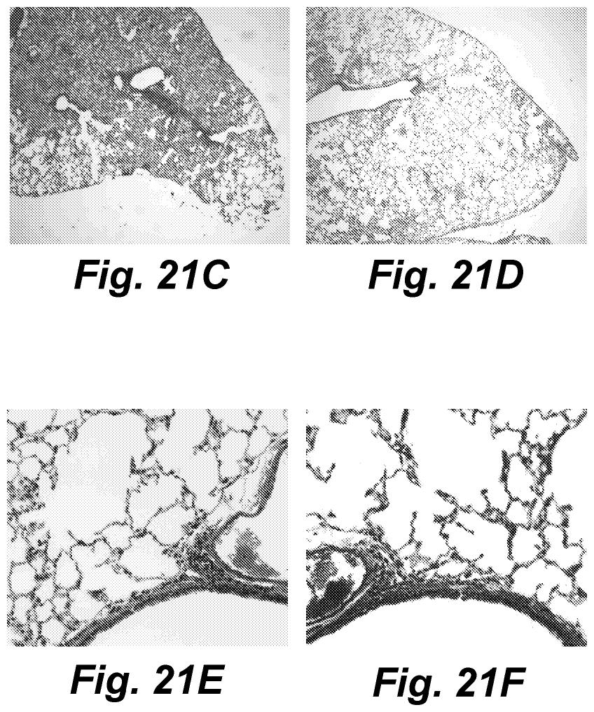

The bactericidal activity of the blue compound was tested against progressive pulmonary tuberculosis using an experimental infection model in BALB/c mice. 8 μg of the blue 1,4 benzoquinone was administered by an intratracheal route every other day for two months; during this time, infected mice showed improvement by not losing weight and not showing piloerection (which are usual signs in mice affected with progressive pulmonary tuberculosis). They also showed a reduction of the lung bacillary load (greater than 90%) in comparison with the negative control (infected mice treated with saline solution alone) (FIG. 21A). This result correlated with histological changes; treated mice showed a twofold decrease of tissue damage (pneumonia) compared to control animals (FIG. 21C-21F). This same dose was administered intratracheally to healthy mice for one month, after which they were sacrificed, and their lungs examined. Their pulmonary histology exhibited only minor inflammatory infiltrates found around the venules. No fibrosis was seen.

Because the 1,4 benzoquinones could serve as lead compounds for new antibiotics, their toxicity to human cell lines was evaluating. The viability of a lung adenocancinoma cell line, A549, which has served as a model of alveolar Type I pulmonary epithelium (Foster et al., (1998) Exp. Cell Res. 243: 359-366; Lin et al., (1998) Infect. Immunity 66: 1121-1126), was tested. In the presence of the red and blue 1,4 benzoquinones at concentrations of 1, 5, and 25 μM, the A549 cells remained relatively unaffected (FIGS. 13A and 13B), suggesting that the direct application of these 1,4 benzoquinones to lungs for the treatment of tuberculosis may be possible. Under the same in vitro conditions, it was found that after a 12-hour culture period, the red and blue 1,4 benzoquinones were comparably potent in inducing death of Jurkat (T-cell leukemia cell line), TE 671 (rhabdomyosarcoma cell line) and SH-SY5Y (bone marrow neuroblastoma cell line) (FIGS. 13A and 13B).

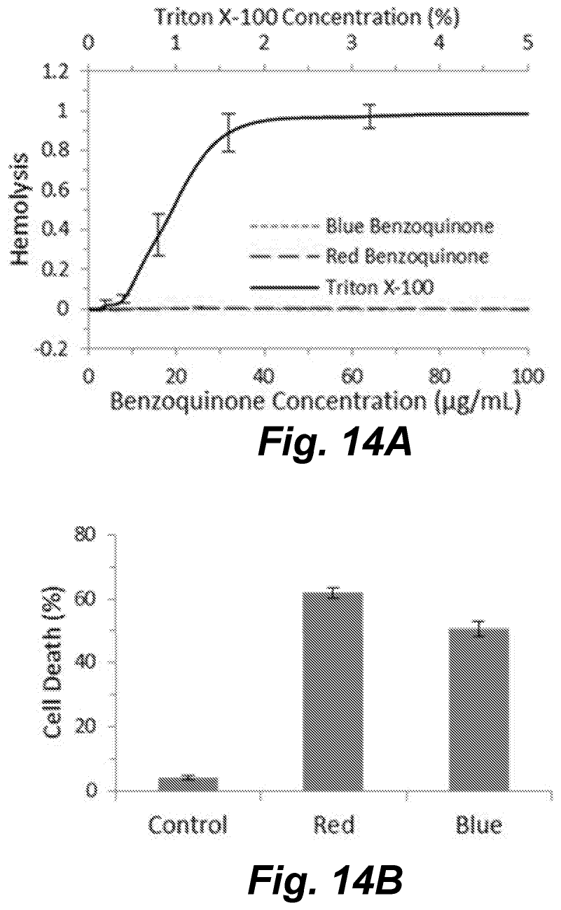

In addition, the red and blue benzoquinones were tested on two types of cells commonly found in human blood, erythrocytes and peripheral blood mononuclear cells (PBMCs). Even at doses higher than 100 μg/mL, no erythrocyte hemolysis was observed after 2 hours (FIG. 14A). However, at a concentration of 25 μM, the blue and red 1,4 benzoquinones killed 50 and 60% of PBMCs, respectively, after 12 hours (FIG. 14B).

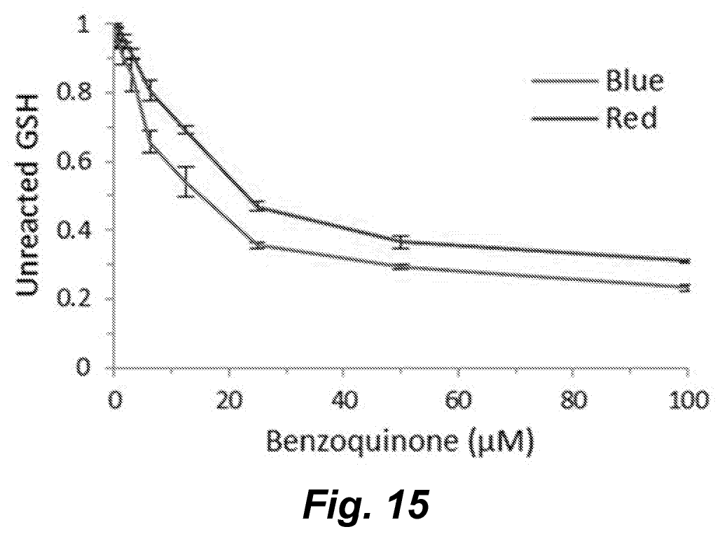

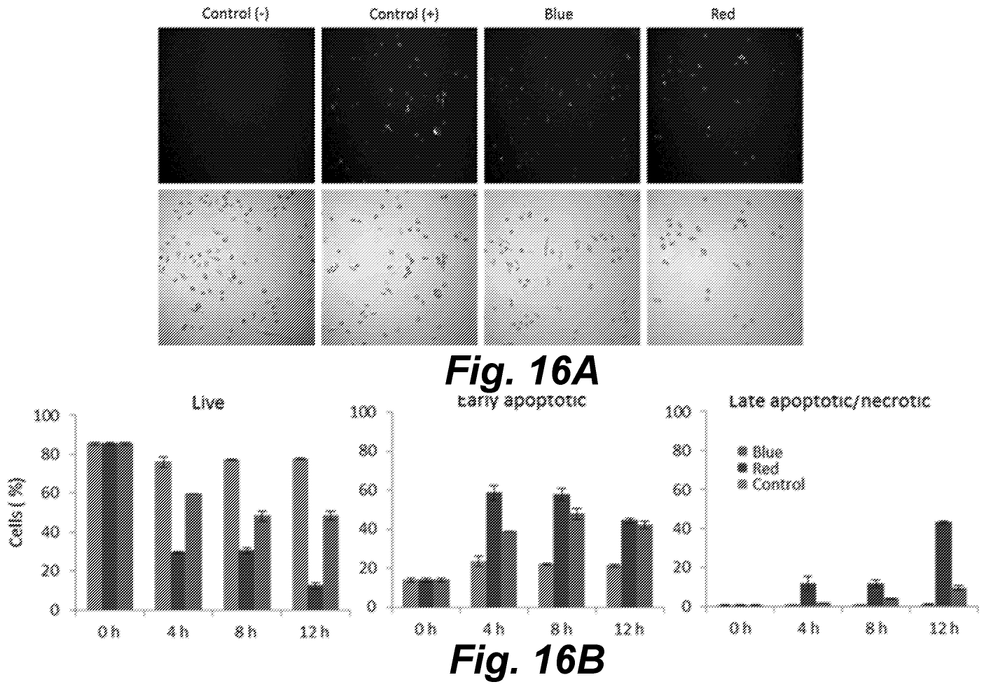

An in vitro glutathione oxidation assay was performed as a first step in investigating the mechanism underlying the marked cytotoxicity of these 1,4-benzoquinone compounds. Both 1,4 benzoquinones oxidized glutathione to the corresponding derivatives in a dose-dependent manner (FIG. 15); this suggests that depletion of glutathione, an important cellular antioxidant, may play a role in triggering cell death (Butler & Hoey (1992) Free Radical Biol. Med. 12(5):337-345). It has also been found that incubation of cells with the 1,4 benzoquinones leads to the formation of reactive oxygen species (ROS) (FIG. 16A) and a time-dependent loss of the cell membrane asymmetry (FIG. 16B), suggesting an apoptotic mode of cell death.

### Formulations

The composition of the present invention can be formulated in a neutral or salt form. Pharmaceutically-acceptable salts, include the acid addition salts and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

A composition of the disclosure may be sterilized by, for example, filtration through a bacteria retaining filter, addition of sterilizing agents to the composition, irradiation of the composition, or heating the composition. Alternatively, the compounds or compositions of the present disclosure may be provided as sterile solid preparations e.g. lyophilized powder, which are readily dissolved in sterile solvent immediately prior to use.

A compound of the disclosure may be formulated into a pharmaceutical composition for administration to a subject by appropriate methods known in the art. Pharmaceutical compositions of the present disclosure or fractions thereof comprise suitable pharmaceutically acceptable carriers, excipients, and vehicles selected based on the intended form of administration, and consistent with conventional pharmaceutical practices. Suitable pharmaceutical carriers, excipients, and vehicles are described in the standard text, Remington: The Science and Practice of Pharmacy (21.sup.st Edition. 2005, University of the Sciences in Philadelphia (Editor), Mack Publishing Company), and in The United States Pharmacopeia: The National Formulary (USP 24 NF19) published in 1999. By way of example for oral administration in the form of a capsule or tablet, the active components can be combined with an oral, non-toxic pharmaceutically acceptable inert carrier such as lactose, starch, sucrose, methyl cellulose, magnesium stearate, glucose, calcium sulfate, dicalcium phosphate, mannitol, sorbital, and the like. For oral administration in a liquid form, the chug components may be combined with any oral, non-toxic, pharmaceutically, acceptable inert carrier such as ethanol, glycerol, water, and the like. Suitable binders (e.g., gelatin, starch, corn sweeteners, natural sugars including glucose; natural and synthetic gums, and waxes), lubricants (e.g. sodium oleate, sodium stearate, magnesium stearate, sodium benzoate, sodium acetate, and sodium chloride), disintegrating agents (e.g. starch, methyl cellulose, agar, bentonite, and xanthan gum), flavoring agents, and coloring agents may also be combined in the compositions or components thereof. Compositions as described herein can further comprise wetting or emulsifying agents, or pH buffering agents.

A formulation or dosage form of the disclosure may be an immediate release dosage form or a non-immediate release delivery system, including without limitation a delayed-release or sustained-release dosage form.

The pharmaceutical compositions of the subject invention can be formulated according to known methods for preparing pharmaceutically useful compositions. Furthermore, as used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations containing pharmaceutically acceptable carriers are described in a number of sources which are well known and readily available to those skilled in the art. For example, Remington's Pharmaceutical Sciences (Martin E W, Remington's Pharmaceutical Sciences, Easton Pa., Mack Publishing Company, 19.sup.th ed., 1995) describes formulations that can be used in connection with the subject invention. Formulations suitable for parenteral administration include, for example, aqueous sterile injection solutions, which may contain antioxidants, buffers, bacteriostats, and solutes which render the formulation isotonic with the blood of the intended recipient; and aqueous and nonaqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question.

The pharmaceutical compositions disclosed herein may be orally administered, for example, with an inert diluent or with an assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of the unit. The amount of active compounds in such therapeutically useful compositions is such that a suitable dosage will be obtained.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, 1,4 benzoquinones of the present disclosure or a pharmaceutically acceptable salt thereof may be incorporated into sustained-release preparation and formulations.

The pharmaceutical forms suitable for injectable use include sterile aqueous solutions or dispersions and sterile powders for the extemporaneous preparation of sterile injectable solutions or dispersions. In all cases of injection, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating 1,4 benzoquinones of the present disclosure, or a pharmaceutically acceptable salt thereof, in the required amount in the appropriate solvent with other various ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

For oral prophylaxis, 1,4 benzoquinones of the present disclosure or a pharmaceutically acceptable salt thereof, may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, 1,4 benzoquinones of the present disclosure or a pharmaceutically acceptable salt thereof, may be incorporated into an antiseptic wash containing sodium borate, glycerin and potassium bicarbonate. 1,4 benzoquinones of the present disclosure or a pharmaceutically acceptable salt thereof may also be dispersed in dentifrices, including: gels, pastes, powders and slurries. 1,4 benzoquinones of the present disclosure or a pharmaceutically acceptable salt thereof may be added in a therapeutically effective amount to a paste dentifrice that may include water, binders, abrasives, flavoring agents, foaming agents, and humectants.

### Methods of Using the Compounds and Formulations Thereof

The compounds and/or formulations described herein can be administered to a subject. The subject can be a subject in need thereof. The subject can have or be suspected of having a bacterial infection such as, but not limited to, a Staphylococcus infection such as a Staphylococcus aureus infection or a Mycobacterial infection such as a Mycobacterial tuberculosis infection. The compounds and formulations described herein can be used to treat a bacterial infection in a subject in need thereof. The compounds and formulations described herein can be administered by a suitable route that delivers the active compound to the desired tissue or cell target. Methods such as, but are not limited to, oral and intravenous administration are advantageous for delivery. Another advantageous delivery route is intratracheal treatment of the lungs such as was used with delivery of the blue compound to the lungs of mice. Usefully, the compounds and formulations of the disclosure have been shown not to be toxic to lung epithelial cells, allowing direct delivery into the lungs. Other suitable routes are described elsewhere herein.

One aspect of the disclosure, therefore, encompasses embodiments of a 1,4-benzoquinone having the structure:

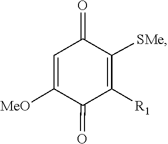

wherein R₁ can be a methylthio group or an alkoxy group.

In some embodiments of this aspect of the disclosure, the 1,4-benzoquinone can have a structure according to Formula A:

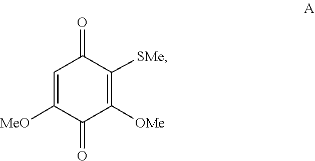 [SMILES: COC1=CC(=O)C(SC)=C(OC)C1=O]

or a derivative thereof.

In some embodiments of this aspect of the disclosure, the 1,4-benzoquinone can have a structure according to Formula B:

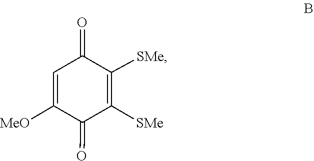 [SMILES: COC1=CC(=O)C(SC)=C(SC)C1=O]

or a derivative thereof.

Another aspect of the disclosure encompasses embodiments of a pharmaceutical formulation comprising: a 1,4-benzoquinone having the structure:

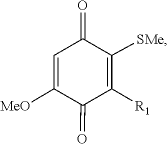

wherein R₁ is a methylthio group or an alkoxy group; and

a pharmaceutically acceptable carrier.

Still another aspect of the disclosure encompasses embodiments of a method of synthesizing a 1,4-benzoquinone, wherein the 1,4-benzoquinone can have a structure according to Formula A:

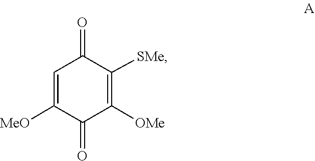 [SMILES: COC1=CC(=O)C(SC)=C(OC)C1=O]

and wherein the 1,4-benzoquinone can be synthesized according to Scheme A:

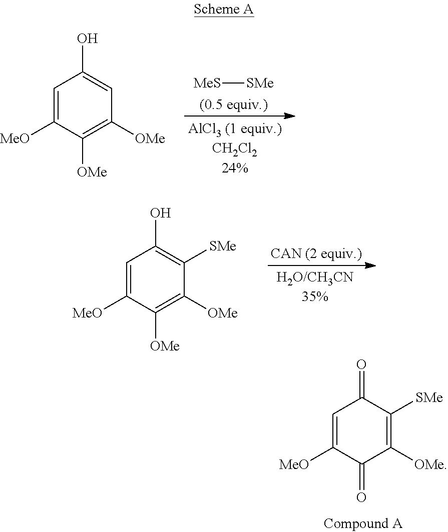

Still another aspect of the disclosure encompasses embodiments of a method of synthesizing a 1,4-benzoquinone, wherein the 1,4-benzoquinone can have a structure according to Formula B:

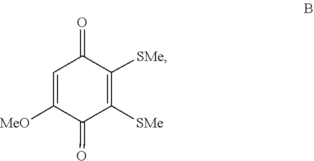 [SMILES: COC1=CC(=O)C(SC)=C(SC)C1=O]

and wherein the 1,4-benzoquinone is synthesized according to Scheme B:

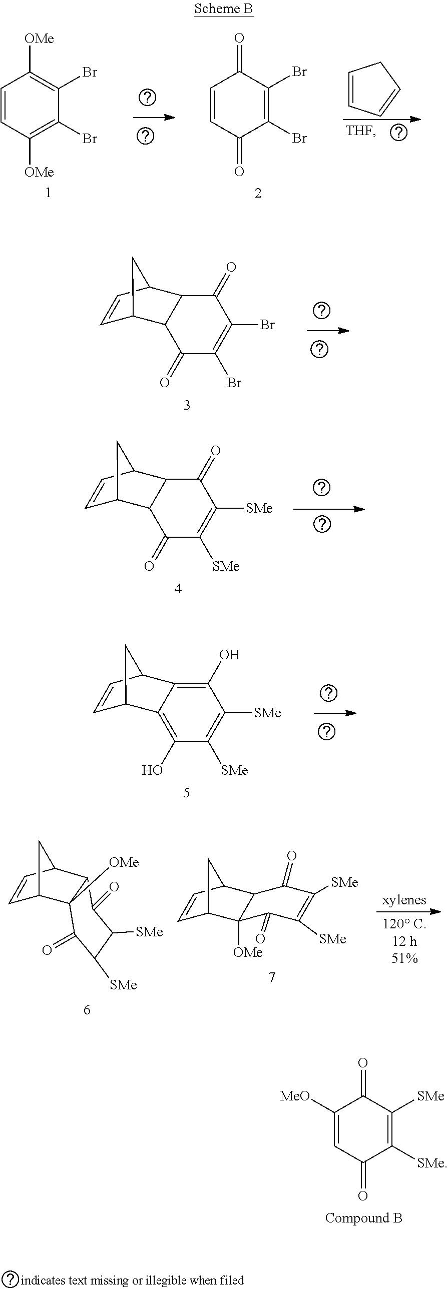

Yet another aspect of the disclosure encompasses embodiments of a method of reducing the proliferation of a bacterial species, the method comprising the step of contacting a population of a bacterial species with an amount of a 1,4-benzoquinone having a structure:

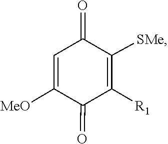

wherein R₁ can be a methylthio group or an alkoxy group and for a period sufficient to reduce the proliferation of the bacterial species.

In some embodiments of this aspect of the disclosure, the bacterial species can be a Staphylococcus or a Mycobacterium.

In some embodiments of this aspect of the disclosure, the bacterial species can be a Staphylococcus aureus or a Mycobacterium tuberculosis.

In some embodiments of this aspect of the disclosure, the 1,4-benzoquinone can be administered to an animal or human subject having a bacterial infection.

In some embodiments of this aspect of the disclosure, the 1,4-benzoquinone can be administered to the animal or human subject in a pharmaceutically acceptable formulation comprising the 1,4-benzoquinone and a pharmaceutically acceptable carrier.

Still another aspect of the disclosure encompasses embodiments of a method of treating a bacterial infection in an animal or human subject, the method comprising: administering to the animal or human subject a pharmaceutically acceptable formulation comprising a 1,4-benzoquinone having the structure:

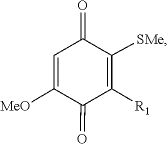

wherein R₁ is a methylthio group or an alkoxy group; and a pharmaceutically acceptable carrier.

In some embodiments of this aspect of the disclosure, the 1,4-benzoquinone has a structure according to Formula A:

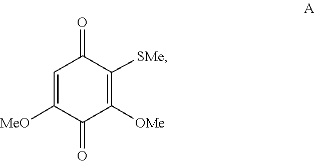 [SMILES: COC1=CC(=O)C(SC)=C(OC)C1=O]

or a derivative thereof.

In some embodiments of this aspect of the disclosure, the bacterial infection can be a Staphylococcal infection.

In some embodiments of this aspect of the disclosure, the bacterial infection can be a Staphylococcus aureus infection.

In some embodiments of this aspect of the disclosure, the 1,4-benzoquinone can have a structure according to Formula B:

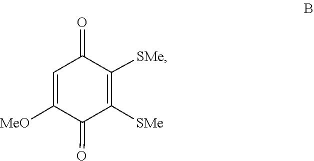 [SMILES: COC1=CC(=O)C(SC)=C(SC)C1=O]

or a derivative thereof.

In some embodiments of this aspect of the disclosure, the bacterial infection can be a Mycobacterial infection.

In some embodiments of this aspect of the disclosure, the bacterial infection can be a Mycobacterium tuberculosis infection.

Another aspect of the disclosure encompasses embodiments of a method of reducing the proliferation of a population of cancer cells, the method comprising the step of contacting a population of cancer cells with an amount of a 1,4-benzoquinone having the structure:

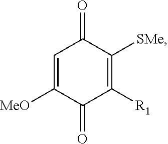

wherein R₁ is a methylthio group or an alkoxy group and for a period sufficient to reduce the proliferation of the cancer cells.

In some embodiments of this aspect of the disclosure, the population of cancer cells is a tumor or a non-tumor cancer.

In some embodiments of this aspect of the disclosure, the population of cancer cells can be a non-tumor cancer, wherein the non-tumor cancer is a leukemia.

In some embodiments of this aspect of the disclosure, the 1,4-benzoquinone can be administered to the animal or human subject in a pharmaceutically acceptable formulation comprising the 1,4-benzoquinone and a pharmaceutically acceptable carrier.

In some embodiments of this aspect of the disclosure, the 1,4-benzoquinone can have a structure according to Formula A or Formula B:

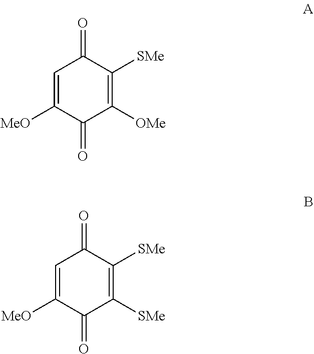 [SMILES: COC1=CC(=O)C(SC)=C(OC)C1=O ; COC1=CC(=O)C(SC)=C(SC)C1=O]

or a derivative thereof.

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### EXAMPLES

### Example 1

Preparation of red and blue 1,4 benzoquinones from the venom of Diplocentrus melici: The venom of Diplocentrus melici scorpions was extracted by electrically stimulating the base of the telson with two-second pulses of 35 V and recovering the viscous colorless venom by capillarity using a pipette tip. Immediately after extraction, the venom from 50 scorpions was exposed to the air until it turned dark red. This red-colored air-exposed venom was suspended in ammonium acetate (20 mM, pH=4.7) and centrifuged for 10 minutes at 14,000×g in order to remove protein aggregates and mucoproteins. The supernatant was then fractionated by gel filtration chromatography using a Sephadex G-50 column (L×I.D., 60 cm×26 mm) with a constant flow of 1 mL/min in ammonium acetate (20 mM, pH=4.7) and monitored by absorbance at 280 nm and 325 nm. Colored fractions were lyophilized and further purified with a C18 analytic reversed-phase HPLC column (Vydac, Hysperia, Calif.), using a linear gradient from 100% of solution A (0.12% trifluoroacetic acid (TFA) in water) to 60% of solution B (0.10% TFA in acetonitrile) over 60 min at a flow rate of 1 mL/min. Purified red 1,4 benzoquinone was dried and stored at −20° C. until use.

### Example 2

Preparation of blue 1,4 benzoquinone from the venom of Diplocentrus melici: Freshly extracted venom was immediately resuspended, with minimal contact with air, in acetone at 4° C. and homogenized by sonication for 30 secs. Immediately after, the solution was centrifuged at 14,000×g for 10 mins at 4° C. The supernatant was separated and freeze-dried (Savant apparatus). The dried powder was resuspended in 0.1% TFA and fractionated on a C18 column (0% to 60% acetonitrile, 60 minutes, Flow Rate=1 mL/min). Colorless fractions were recovered and dried. They were then resuspended in water and bubbled with air for two hours to allow oxidation of precursor compounds. The treated fractions were analyzed by RP-HPLC to isolate the blue 1,4 benzoquinone. Purified blue 1,4 benzoquinone was dried and stored at −20° C. until use.

### Example 3

Structural characterization of the red and blue benzoquinones: Electrospray ionization mass spectrometric (ESI-MS) studies were performed on a high-resolution mass spectrometer (Thermo Scientific LTQ Orbitrap XL Hybrid Ion Trap-Orbitrap mass spectrometer) using a homebuilt ESI source. Nitrogen (120 psi) was used as a sheath gas. Electrospray of the analyte solution was performed in positive (+5 kV) or negative (−5 kV) ion mode. The heated capillary (MS inlet) temperature and voltage were maintained at 275° C. and 44 V respectively. Helium was used as the collision gas in the collision induced dissociation cell (CID cell; an ion trap). CID spectra (MS/MS) were acquired using an isolation width of 0.9 m/z unit with activation Q and activation time set to 0.25 and 30 ms, respectively. All experiments were carried out under identical conditions, unless otherwise stated. The ion optics were tuned to get maximum ion count. Data acquisition was performed using XCalibur software (Thermo Fisher Scientific).

Nuclear magnetic resonance (NMR) spectra were acquired on either a Varian Inova-600 operating at 600 and 150 MHz, a Varian Inova-300 operating at 300 and 75 MHz, a Varian Mercury-400 operating at 400 and 100 MHz, or a Varian Inova-500 operating at 500 and 125 MHz, and referenced internally according to residual solvent signals. Data for NMR were recorded as follows: chemical shift (δ, ppm), multiplicity (s, singlet; br s, broad singlet; d, doublet; t, triplet; q, quartet; quint, quintet; sext, sextet; m, multiplet), integration, coupling constant (Hz). Data are reported in terms of chemical shift (δ, ppm). Infrared spectra were recorded on either a Thermo-Nicolet IR100 spectrometer or a Thermo-Nicolet IR300 spectrometer as thin films using NaCl salt plates and are reported in frequency of absorption.

### Example 4

Chemical synthesis of red and blue 1,4 Benzoquinones: All reagents were obtained commercially unless otherwise noted. Reactions were performed using glassware that was oven-dried. Air- and moisture-sensitive liquids and solutions were transferred via syringe or stainless steel cannula. Organic solutions were concentrated under reduced pressure (˜15 Torr) by rotary evaporation. Solvents were purified by passage under 12 psi through activated alumina columns.

Chromatography was performed on Silicycle Silia-P Silica Gel (40-63 μm).

Compounds purified by chromatography were typically applied to the adsorbent bed using the indicated solvent conditions with a minimum amount of added methylene chloride as needed for solubility. Thin layer chromatography was performed on either Whatman Partisil K6F Silica Gel 60 Å plates (250 μm) or EMD Chemicals Silica Gel (250 μm). Visualization of the developed chromatogram was accomplished by fluorescence quenching and/or by staining with butanolic ninhydrin, aqueous potassium permanganate, aqueous ceric ammonium molybdate (CAM), or ethanolic anisaldehyde.

### Example 5

Cytotoxicity to PBMCs and to Neoplastic Cells: The cytotoxicity of the 1,4 benzoquinones of the disclosure was tested with peripheral blood mononuclear cells (PBMCs) and various human neoplastic cell lines: A549 (adenocarcinomic lung epithelial cells), Jurkat (T cell leukemia), TE 671 (rhabdomyosarcoma cells) and SH-SY5Y (bone marrow neuroblastoma cells).

PBMCs were obtained from healthy blood donors and isolated by Ficoll-Paque PLUS density gradient centrifugation and resuspended in RPMI-1640 medium supplemented with 10% fetal calf serum. Cells were incubated at 37° C. in 5% CO₂ overnight and non-adherent cells were recovered and used for subsequent experiments. The neoplastic cell lines were cultured according to the ATCC guidelines.

For cytotoxicity assays, cells were resuspended in DMEM without phenol red, supplemented with 2% heat-inactivated fetal calf serum. Cells were plated in 96-well polystyrene cell culture plates (2×10⁴ to 2×10⁵ cells/well depending on the cell type) and incubated at 37° C. in 5% CO₂ for 8 hours to promote cell adhesion to the well's surface. The red and blue 1,4 benzoquinones were administered to achieve final concentrations of 1, 5, and 25 μM. Cells were then incubated at 37° C. in 5% CO2 for 12 hours. Following the incubation period, plates were centrifuged at 300×g for 10 minutes and supernatants were harvested to evaluate cell viability with the CytoTox 96@ Non-Radioactive Cytotoxicity Assay (Promega). This assay quantitatively measures lactate dehydrogenase (LDH), a stable cytosolic enzyme that is released upon cell lysis. The released LDH in the culture supernatants was measured with a colorimetric coupled enzymatic assay according to the manufacturer's instructions.

### Example 6

Hemolysis assay: The hemolytic activity was evaluated for both of the 1,4 benzoquinones using fresh human erythrocytes. Fresh human erythrocytes were washed four times with phosphate buffered saline (PBS) and incubated for 2 h at 37° C. with different concentration of the 1,4 benzoquinones. A gradient of Triton X-100 was used as positive control of hemolysis. After incubation, the erythrocytes suspension was centrifuged at 400×g for 10 min and supernatant was recovered. Hemolysis was determined by the absorbance of the supernatant at 415 nm.

### Example 7

Glutathione oxidation assay: To elucidate the mechanism of inhibitory action of both benzoquinones, the reactivity with the reduced form of L-glutathione (GSH) was evaluated in a colorimetric assay. First, both components were diluted to different concentrations (0-100 μM) in PBS (pH, 7.4) and then reacted with 120 μM of glutathione. Reaction was incubated for 1 h at 37° C. and the remaining GSH (not oxidized) was reacted with 200 μM of the sulfhydryl reagent 5,5′-dithio-bis(2-nitrobenzoic acid) (DTNB) to form a yellow derivative measurable at 412 nm. Reaction products were evaluated by HPLC-MS.

### Example 8

TABLE 1
Minimum inhibitory concentration of the Red and the Blue Compounds
against clinical isolates of methicillin-resistant S. aureus (MRSA)

| | | μg/ml | | | | | | | | MIC |
|---|---|---|---|---|---|---|---|---|---|---|
| Extract | Strain | 0.5 | 1 | 2 | 4 | 8 | 16 | 32 | 64 | (μg/ml) |
| Red | Staphylococcus | R | S | S | S | S | S | S | S | 1 |
| | aureus | | | | | | | | | |
| | ATCC 29213 | | | | | | | | | |
| | 09 3001 | R | R | R | R | R | R | R | R | >64 |
| | 21 2001 | R | R | R | R | R | R | R | R | >64 |
| | 01 2104 | R | R | R | R | R | R | R | R | >64 |
| | 01 2075 | R | R | R | R | R | R | R | R | >64 |
| Blue | Staphylococcus | R | S | S | S | S | S | S | S | 1 |
| | aureus | | | | | | | | | |
| | ATCC 29213 | | | | | | | | | |
| | 09 3001 | R | R | R | R | R | R | R | R | >64 |
| | 21 2001 | R | R | R | R | R | R | R | R | >64 |
| | 01 2104 | R | R | R | R | R | R | R | R | >64 |
| | 01 2075 | R | R | R | R | R | R | R | R | >64 |

S Sensitive (did not grow the crop); R resistant (bacterial growth)

TABLE 2
Time-kill assay of 1,4-Benzoquinones (red and
blue compounds) from Diplocentrus
melici against Staphylococcus aureus.
Colony-forming Units (CFU)^{b}

| Time | | MIC | 2 × MIC | MIC | 2 × MIC |
|---|---|---|---|---|---|
| (hr)a | S. aureus | red | red | blue | blue |
| 0 | 315 | 298 | 311 | 352 | 339 |
| 6 | >600 | 22 | 9 | 45 | 21 |
| 12 | countless | 3 | 0 | 1 | 2 |
| 24 | countless | 0 | 0 | 2 | 0 |

aIncubation time in the broth microdilution assay.
^{b}Colony-forming units per plate. A sample of 100 pL of a 1:200 dilution of a 0.5

McFarland standard of bacterial culture was evenly spread on a Mueller Hinton agar plate.

### Example 9

### Synthesis of Red Compound A (3,5-dimethoxy-2-(methylthio)cyclohexa-2,5-diene-1,4-dione)

### i) Synthesis of Compound 2

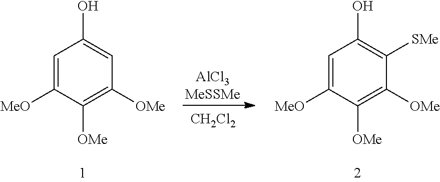

Compound 1 (0.450 g, 2.47 mmol) was dissolved in 15 mL of CH₂Cl₂. MeSSMe (0.110 mL, 1.24 mmol) was added to this solution followed by anhydrous AlCl₃ (0.329 g, 2.47 mmol). After 30 minutes at room temperature, the reaction was quenched by slow, careful addition of 1M HCl. The contents of the reaction flask were transferred to a separatory funnel with 20 mL of H₂O and 20 mL of CH₂Cl₂. The layers were separated, and the aqueous fraction was extracted with an addition 2×20 mL of CH₂Cl₂. The organic layers were combined, dried with Na₂SO₄, and concentrated under reduced pressure to yield compound 2 as a light yellow oil. ¹H NMR (500 MHz, CDCl₃) δ 6.85 (s, 1H), 6.39 (s, 1H), 3,99 (s, 3H), 3.85 (s, 3H), 3.81 (s, 3H), 2.26 (s, 3H) ppm. ¹³C NMR (125 MHz, CDCl₃) δ 155.8, 1553, 153.9, 136.2, 105.4, 94.4, 61.8, 61.4, 56.2, 19.5 ppm. IR (thin film) v 3384, 2850, 1463, 1111 cm⁻¹ HRMS (ES⁺) calcd for C₁₀H₁₅O₄S⁺ 231.0686 found 231.0698 (MH⁺).

### ii) Synthesis of Compound 3 (Red Compound)

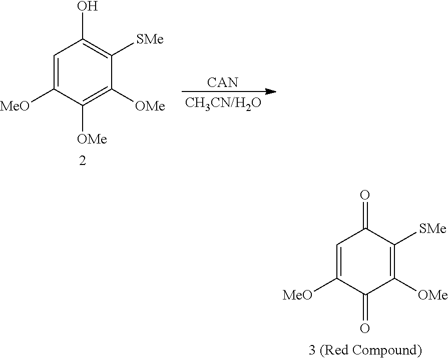

Compound 2 (0.138 g, 0.600 mmol) was dissolved in 10 mL of CH₃CN. An aqueous solution (2 mL) of Ceric Ammonium Nitrate (CAN) (0.657 g, 1.20 mmol) was added dropwise. After stirring for 30 min at room temperature, the contents of the reaction flask were transferred to a separatory funnel with 20 mL of H₂O and 20 mL of CH₂Cl₂. The layers were separated and the aqueous layer was extracted with 1×20 mL of CH₂Cl₂ and 1×20 mL of EtOAc. The organic fractions were combined, dried with Na₂SO₄, and concentrated under reduced pressure. Purification by chromatography on silica gel followed by recrystallization from 1:4 EtOAc/hexanes yielded 3 (45 mg, 0.210 mmol) as a red solid. ¹H NMR (500 MHz, Acetone-d₆) δ 5.95 (s, 1H), 3.94 (s, 3H), 3.83 (s, 3H), 2.55 (s, 3H), ¹³C NMR (126 MHz, Acetone-d₆) δ 184.94, 175.85, 159.14, 134.55, 107.81, 61.08, 57.01, 16.36.

### Example 10

### Synthesis of Blue Compound B (5-methoxy-2,3-bis(methylthio)cyclohexa-2,5-diene-1,4-dione)

### i) Synthesis of Compound 5

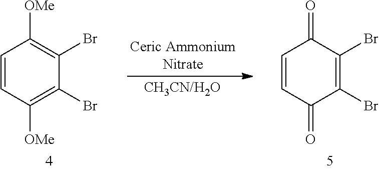 [SMILES: COc1ccc(OC)c(Br)c1Br ; O=C1C=CC(=O)C(Br)=C1Br]

4 mL of an aqueous solution of Ceric Ammonium Nitrate (3.70 g, 6.76 mmol) were added dropwise to a vigorously stirring solution of compound 4 (0.500 g, 1.70 mmol) in 20 mL of CH₃CN. After 1 hour at room temperature, the contents of the reaction flask were transferred to separatory funnel with 20 mL of H₂O and 40 mL of CH₂Cl₂. The layers were separated, and the organic fraction collected. The aqueous layer was extracted under reduced pressure to a yellow solid (0.460 g) that was used directly in the next reaction. 5 is a known compound and spectral data matched that which is reported in Synthetic Communication, 1999, vol. 29: 821-825.

### ii) Synthesis of Compound 6

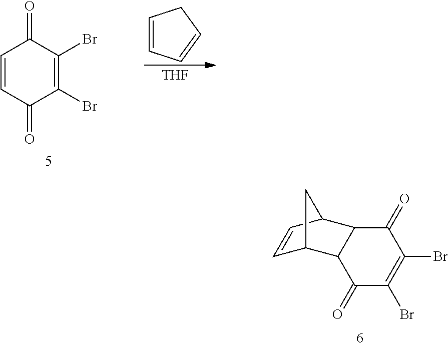 [SMILES: O=C1C=CC(=O)C(Br)=C1Br ; C1=CCC=C1 ; O=C1C(Br)=C(Br)C(=O)C2C3C=CC(C3)C12 ; "THF"]

To a solution of compound 5 (0.460 g, 1.73 mmol) in 10 mL if THF was added to 2 mL of freshly distilled cyclopentadiene. After stirring for 5 h at room temperature, the solution was concentrated under reduced pressure to yield 0.326 g of an inseparable mixture of compound 6 and dicyclopentadiene. This mixture was used directly in the next step.

### iii) Synthesis of Compound 7

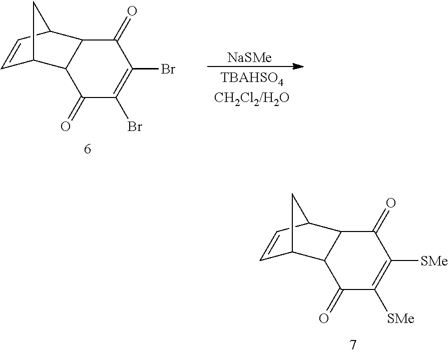 [SMILES: O=C1C(Br)=C(Br)C(=O)C2C3C=CC(C3)C12 ; CSC1=C(SC)C(=O)C2C3C=CC(C3)C2C1=O ; "NaSMe"]

Compound 7 was prepared by the method of Ferreira et al., ((2003) Tetrahedron 59: 1349-1357, incorporated herein by reference). Briefly, compound 6 (0.326 g, 1.22 mmol) was dissolved in 10 mL of CH₂Cl₂ and transferred to a separatory funnel. 10 mL of an aqueous solution containing NaSMe (0.170 g, 2.40 mmol) and tetrabutylammonium hydrogensulfate (0.025 g, 0.073 mmol) were added to the separatory funnel. The funnel was capped and shaken for approximately two minutes. The phases were separated, and the organic layer was collected. The aqueous fraction was extracted with an additional 20 mL of CH₂Cl₂. The organic fractions were pooled, dried with Na₂SO₄, and concentrated under reduced pressure to a yellow solid, which was used directly in the next step.

### iv) Synthesis of Compound 8

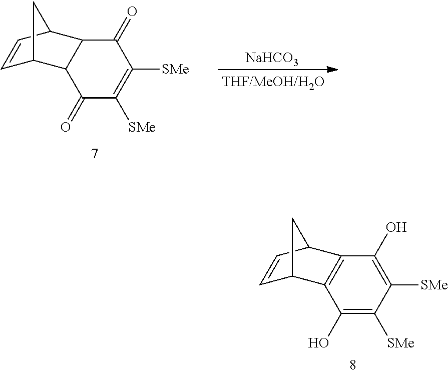

Compound 8 was prepared in a procedure similar to that reported by Wladislaw et al., Synthesis, 1983: 464-466). Briefly, compound 7 was dissolved in a 1:1:1 mixture of THF, MeOH, and H₂O, and 5 equivalents of NaHCO₃ were added to this solution. The resulting mixture was heated at 70° C. for 3 h. After cooling to room temperature, the suspension was acidified with 1M HCl and transferred to a separatory funnel with 20 mL of H₂O and 30 mL of CH₂Cl₂. The layers were separated, and the organic fraction was collected. The aqueous fraction was extracted with 1×20 mL of CH₂Cl₂ and 1×20 mL of EtOAc. The organic extracts were pooled, dried with Na₂SO₄, and concentrated under reduced pressure. Purification by chromatography on silica gel yielded compound 8.

### v) Synthesis of Compound 9

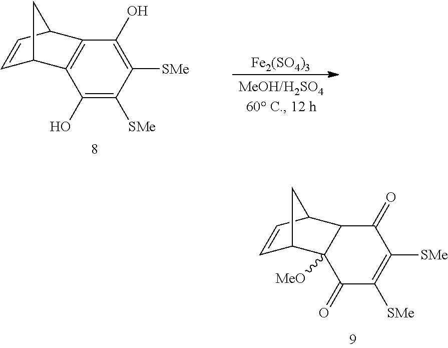

Compound 8 (0.365 g, 1.37 mol) was dissolved in a mixture of MeOH (20 mL) and H₂SO₄ (0.5 mL), Fe₂(SO₄)₃ (0.550 g, 1.37 mmol) was added and the resulting suspension was heated to 60° C. for 12 h. The color of the suspension first became dark red and then transitioned to dark yellow over this period. After cooling to room temperature, this solution was transferred to a separatory funnel with 20 mL of H₂O and 20 mL of CH₂Cl₂. The layers were separated, and the aqueous fraction was extracted with 2×20 mL of CH₂Cl₂. The organic fractions were combined, dried with Na₂SO₄, and concentrated under reduced pressure. Purification by chromatography on silica gel yielded a yellow solid (compound 9, 0.250 g) as a mixture of diastereomers (9 and 9′ in Scheme 2).

### vi) Synthesis of Compound 10

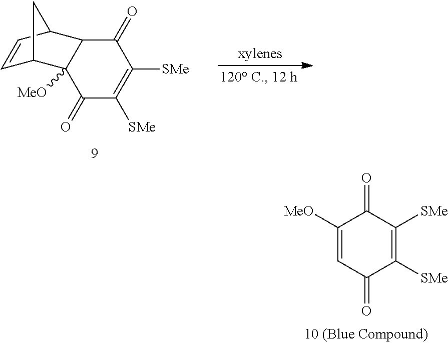 [SMILES: COC12C(=O)C(SC)=C(SC)C(=O)C1C1C=CC2C1 ; COC1=CC(=O)C(SC)=C(SC)C1=O]

Compound 9 (0.250 g, 0.843 mmol) was dissolved in xylenes (10 mL), and this solution was heated to 120° C. for 12 h in a flask closed with a septum pierced with a needle, which has left open to the atmosphere. Purification by chromatography on silica gel yielded compound 10 as a blue solid (0.100 g, 0.434 mmol). ¹H NMR (300 MHz, CDCl₃) δ 5.90 (s. 1H), 3.83 (s, 3H), 2.70 (s, 3H), 2.60 (s, 3H) ppm; ¹³C NMR (75 MHz, CDCl₃) δ 181.3, 175.5, 159.4, 1483, 140.5, 1083, 56.8, 18.9, 18.1 ppm; IR (thin film) v 2919, 1630, 1440, 1108 cm⁻¹; HRMS (ES⁺) calcd for CH₁₁O₃S₂+231.0144 found 231.0148 (MH⁺).

What is claimed:

1. A 1,4-benzoquinone having the structure: 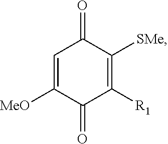
wherein R₁ is a methylthio group or an alkoxy group.

2. The 1,4-benzoquinone of claim 1 having a structure according to Formula A: 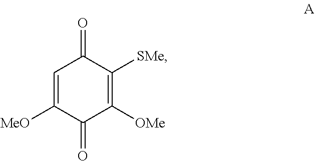 [SMILES: COC1=CC(=O)C(SC)=C(OC)C1=O]
or a derivative thereof.

3. The 1,4-benzoquinone of claim 1 having a structure according to Formula B: 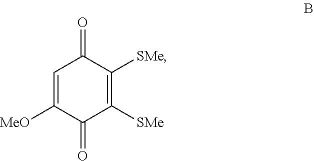 [SMILES: COC1=CC(=O)C(SC)=C(SC)C1=O]
or a derivative thereof.

4. A pharmaceutical formulation comprising:
a 1,4-benzoquinone having the structure: 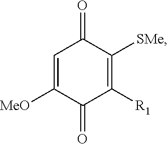
wherein R₁ is a methylthio group or an alkoxy group; and
a pharmaceutically acceptable carrier.

5. A method of synthesizing a 1,4-benzoquinone, wherein the 1,4-benzoquinone has a structure according to Formula A: 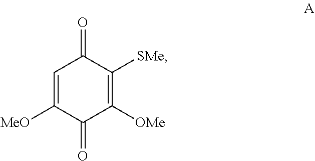 [SMILES: COC1=CC(=O)C(SC)=C(OC)C1=O]
and wherein the 1,4-benzoquinone is synthesized according to Scheme A:

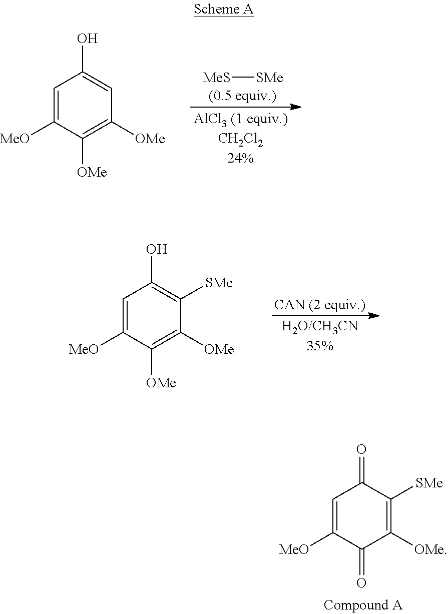

6. A method of synthesizing a 1,4-benzoquinone, wherein the 1,4-benzoquinone has a structure according to Formula B: 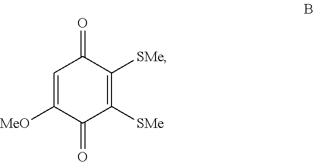 [SMILES: COC1=CC(=O)C(SC)=C(SC)C1=O]
and wherein the 1,4-benzoquinone is synthesized according to Scheme B:

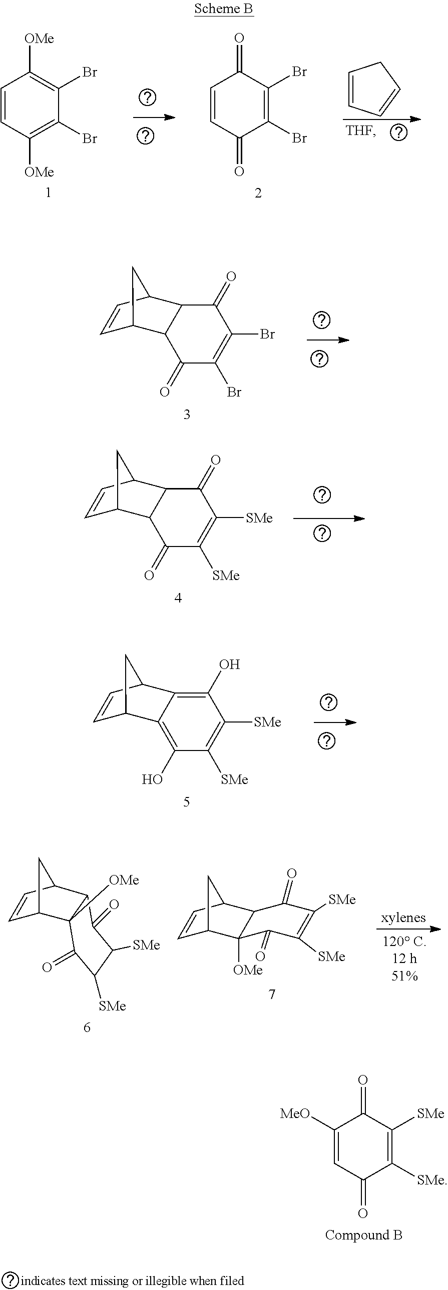

7. A method of reducing the proliferation of a bacterial species, the method comprising the step of contacting a population of a bacterial species with an amount of a 1,4-benzoquinone having a structure: 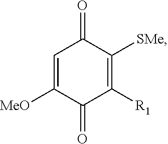
wherein R₁ is a methylthio group or an alkoxy group and for a period sufficient to reduce the proliferation of the bacterial species.

8. The method of claim 7, wherein the bacterial species is a Staphylococcus or a Mycobacterium.

9. The method of claim 8, wherein the bacterial species is a Staphylococcus aureus or a Mycobacterium tuberculosis.

10. The method of claim 7, wherein the 1,4-benzoquinone is administered to an animal or human subject having a bacterial infection.

11. The method of claim 10, wherein the 1,4-benzoquinone is administered to the animal or human subject in a pharmaceutically acceptable formulation comprising the 1,4-benzoquinone and a pharmaceutically acceptable carrier.

12. A method of treating a bacterial infection in an animal or human subject, the method comprising:
administering to the animal or human subject a pharmaceutically acceptable formulation comprising a 1,4-benzoquinone having the structure: 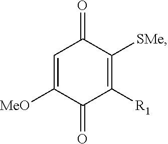
wherein R₁ is a methylthio group or an alkoxy group; and
a pharmaceutically acceptable carrier.

13. The method of claim 12, wherein the 1,4-benzoquinone has a structure according to Formula A: 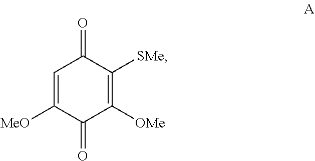 [SMILES: COC1=CC(=O)C(SC)=C(OC)C1=O]
or a derivative thereof

14. The method of claim 13, wherein the bacterial infection is a Staphylococcal infection.

15. The method of claim 14, wherein the bacterial infection is a Staphylococcus aureus infection.

16. The method of claim 12, wherein the 1,4-benzoquinone has a structure according to Formula B: 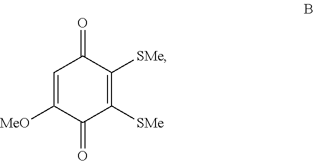 [SMILES: COC1=CC(=O)C(SC)=C(SC)C1=O]
or a derivative thereof.

17. The method of claim 16, wherein the bacterial infection is a Mycobacterial infection.

18. The method of claim 17, wherein the bacterial infection is a Mycobacterium tuberculosis infection.

19. A method of reducing the proliferation of a population of cancer cells, the method comprising the step of contacting a population of cancer cells with an amount of a 1,4-benzoquinone having the structure: 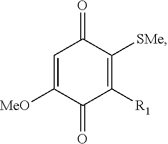
wherein R₁ is a methylthio group or an alkoxy group and for a period sufficient to reduce the proliferation of the cancer cells.

20. The method of claim 19, wherein the population of cancer cells is a tumor or a non-tumor cancer.

21. The method of claim 19, wherein the population of cancer cells is a non-tumor cancer, wherein the non-tumor cancer is a leukemia.

22. The method of claim 19, wherein the 1,4-benzoquinone is administered to the animal or human subject in a pharmaceutically acceptable formulation comprising the compound and a pharmaceutically acceptable carrier.

23. The method of claim 19, wherein the 1,4-benzoquinone has a structure according to Formula A or Formula B: 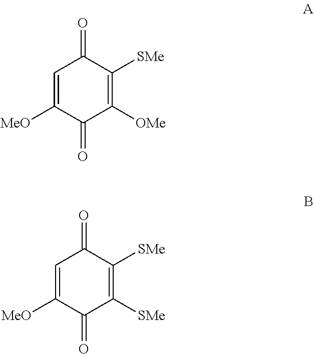 [SMILES: COC1=CC(=O)C(SC)=C(OC)C1=O ; COC1=CC(=O)C(SC)=C(SC)C1=O]
or a derivative thereof.
